(19) United States
(12) Patent Application Publication
(10) Pub. No.: US 2024/0067974 A1
(43) Pub. Date: Feb. 29, 2024

(54) METHOD OF ALLELE SPECIFIC SILENCING FOR THE TREATMENT OF AUTOSOMAL DOMINANT CATECHOLAMINERGIC POLYMORPHIC VENTRICULAR TACHYCARDIA (CPVT)

(71) Applicant: Istituti Clinici Scientifici Maugeri SpA SB, Pavia (IT)

(72) Inventors: Silvia G. PRIORI, Milano (IT); Rossana BONGIANINO, Cura Carpignano (IT); Marco DENEGRI, Stradella (IT); Carlo NAPOLITANO, Milano (IT)

(21) Appl. No.: 18/175,401

(22) Filed: Feb. 27, 2023

Related U.S. Application Data
(62) Division of application No. 16/077,835, filed on Aug. 14, 2018, now Pat. No. 11,591,602.
(60) Provisional application No. 62/295,168, filed on Feb. 15, 2016.

(51) Int. Cl. C12N 15/113; A61K 9/127; A61P 9/00; C12N 15/86

(52) U.S. Cl. CPC C12N 15/1138; A61K 9/127; A61P 9/00; C12N 15/1136; C12N 15/1137; C12N 15/86; C12N 2310/14; C12N 2310/315; C12N 2310/321; C12N 2310/322; C12N 2320/32; C12N 2320/34; C12N 2750/14043; C12N 2750/14071

(57) ABSTRACT

The present invention provides a method for the treatment of autosomal dominant Catecholaminergic Polymorphic Ventricular Tachycardia associated with mutations in the cardiac ryanodine receptor type 2 (RYR2) gene, by the use of an AAV mediated RNA interference approach to induce allele specific silencing of mutant mRNA.

### FIELD OF THE INVENTION

The present invention concerns a method for the treatment of autosomal dominant Catecholaminergic Polymorphic Ventricular Tachycardia, associated with mutations in the cardiac ryanodine receptor type 2 (RYR2) gene, by the use of an AAV mediated RNA interference approach to induce allele specific silencing of mutant mRNA.

### STATE OF THE ART

Catecholaminergic Polymorphic Ventricular Tachycardia (CPVT) is an inherited channelopathy characterized by high susceptibility to life threatening arrhythmias. Two forms of the disease have been described: the autosomal dominant and the autosomal recessive variant. The first is associated with mutations in the cardiac ryanodine receptor type 2 (RYR2) gene (Priori S G et al., 2001), while the autosomal recessive variant is associated with mutations in the cardiac calsequestrin 2 (CASQ2) gene (Lahat H et al., 2001). Clinical observations have shown that patients with the dominant form of CPVT develop bidirectional and polymorphic ventricular tachycardia in response to sympathetic activation, whereas their resting ECGs are unremarkable and heart structure is preserved. The response to current therapy is unable to effectively reduce sudden death in affected individual and therefore there is need for an innovative treatment able to correct all aspects of the functional derangements observed in the dominant form of CPVT.

The pathology is linked to an abnormal function of the physiologic mechanism called ‘calcium-induced calcium release’ (CICR) that is the fundamental for the excitation-contraction coupling in the heart.

The highly coordinated opening and closing of voltage-dependent ion channels located in the membrane of cardiac myocytes generates the cardiac action potential. During the plateau phase of the action potential, opening of voltage-dependent L-type Ca²⁺ channels allows the influx of Ca²⁺ in the plasmalemma. This process triggers the calcium transient and induces opening of sarcoplasmic reticulum (SR) Ca²⁺ release channels: the ryanodine receptor 2 (RyR2) (Bers D M, 2002). These local releases occur at specialized structures called the calcium release units (CRUs). The CRUs are preferentially localized at the level of the transverse tubules (T-tubules), where the membrane of the SR is juxtaposed to the cellular membrane. One CRU is formed by clusters of RyR2 (spanning the SR membrane) that are in close proximity to the L-type Ca²⁺ channels (on the cell membrane) (Franzini-Armstrong et al., 2005). The Ca²⁺ released from the SR binds to troponin C and induces a series of allosteric changes in the myosin filaments leading to muscle fiber contraction. The subsequent removal of Ca²⁺ is mediated by the concomitant closing of the RyR2 and the action of SR Ca²⁺ ATPase (SERCA) that pumps Ca²⁺ back into the SR stores.

Another component of Ca²⁺-transient termination is the Na⁺-Ca²⁺ exchanger (NCX). The NCX extrudes one Ca²⁺ ion (two positive charges) for every three Na⁺ ions (three positive charges) taken into the cell. Thus, the NCX removes Ca²⁺ by generating a net inward depolarizing current: the transient inward current (Iti) (Pieske B et al., 1999). The NCX becomes very important for the removal of Ca²⁺ in conditions characterized by calcium overload, for example in case of RYR2 genetic mutations.

Arrhythmias in CPVT are elicited by Ca²⁺ release events that are not triggered by an action potential and are, therefore, called ‘spontaneous calcium releases’ (SCRs). SCR begins as a localized event involving a single CRU, but can also diffuse to neighboring CRUs triggering more Ca²⁺ release to produce a cell-wide calcium wave. The probability that SCR will lead to a calcium wave is influenced by the balance between SR Ca²⁺ content and the concentration of Ca²⁺ that induces Ca²⁺ release from the SR, the so-called SR calcium threshold. RyR2 function has a pivotal role in controlling the threshold. Several RYR2 mutations associated with CPVT decrease the SR threshold for the release of calcium from the SR and therefore they facilitate the occurrence of Spontaneous Calcium Release (Venetucci L et al., 2012).

When abnormal Ca²⁺ release occurs, cytosolic Ca²⁺ concentration transliently increases and the cell must activate mechanisms to prevent disruption of Ca²⁺ homeostasis and re-establish the physiological diastolic level of Ca²⁺. Extrusion of Ca²⁺ through the NCX is the preferred modality to reduce cytosolic Ca²⁺ however to extrude 1 Ca²⁺ the NCX brings inside the cell 3 Na+ thus creating a net inward current called Transient Inward Current or Iti. This current produces a transient membrane depolarizations known as delayed afterdepolarization (DAD). When a DAD's amplitude reaches the voltage threshold for the opening of the voltage dependent Na⁺ channel, a ‘triggered’ action potential is generated. Propagation of an action potential to the entire heart generates an extrasystolic beat. When this chain of events becomes repetitive and several DADs reach the threshold for the generation of propagating action potentials, triggered arrhythmic activity is elicited and it generates complex and life threatening arrhythmias. Mutations of RYR2 have been shown to facilitate the occurrence of Spontaneous Calcium Releases during β-adrenergic stimulation and, in turn, elicit DADs and triggered activity leading to severe ventricular arrhythmias (Liu N et al., 2006).

The generation and characterization of RyR2 ^{R4496C/+} knock-in mouse model for autosomal dominant CPVT (Cerrone M et al., 2005; U.S. Pat. No. 7,741,529 B1) has provided great insight into the pathogenic mechanisms underlying this disease. RyR2 ^{R4496C/+} heterozygous mice recapitulate human CPVT and develop adrenergically induced bidirectional and polymorphic ventricular arrhythmias. R4496C mutation increases the sensitivity of RyR2 channel to luminal calcium thus facilitating the spontaneous release of calcium from the Sarcoplasmic Reticulum. Spontaneous calcium release begins as a localized event involving a single CRU, however it may also propagate to neighboring CRUs triggering more Ca²⁺ release to produce a cell-wide calcium wave. The probability that SCR will lead to a calcium wave is influenced by the balance between SR Ca²⁺ content and the concentration of Ca²⁺ that induces Ca²⁺ release from the SR, the so-called SR calcium threshold. RyR2 function has a pivotal role in controlling the threshold.

### SUMMARY OF THE INVENTION

The present invention concerns a method for the treatment of autosomal dominant Catecholaminergic Polymorphic Ventricular Tachycardia through silencing sequences that allow to differentiate the normal allele from the diseased allele of the RyR2 gene.

The method for the treatment of autosomal dominant atecholaminergic Polymorphic Ventricular Tachycardia according to the invention comprises the exploitation of therapeutic post-transcriptional gene-silencing. The inventors have found that, taking advantage of the endogenous RNA interference (RNAi) pathway (Elbashir et al., 2001), through the delivery of an artificial miRNA expressing vector into a cardiac cell, it is possible to selectively suppress the expression of mutant RyR2 mRNA leaving almost unaltered the expression of the wild type RyR2 transcript in order to correct functional derangements observed in RyR2 ^{R4496C/+} heterozygous subjects.

The development of an RNAi approach involves some risk such as the supraphysiologic expression of interfering RNAs species and the possibility to cause haploinsufficiency of vital genes, as it is precisely RYR2. Nevertheless, through the accurate selection of interfering RNAs sequences and by using suitable AAV serotype, promoter, as well as vector dose, it is possible to achieve an extent of mutated allele gene silencing that is sufficient to elicit the desired effect, i.e. protecting cardiomyocytes against developing adrenergically triggered activity, but not to affect normal cardiac function. In order to achieve this goal, only strong efficient and strictly specific molecules, derived from the initial in vitro screening, are provided for the use in the in vivo experiments.

### DETAILED DESCRIPTION OF THE INVENTION

The present invention concerns a method for the treatment of autosomal dominant Catecholaminergic Polymorphic Ventricular Tachycardia associated with RYR2 (NM_023868.2, NM_001035.2) mutations in mouse models or in human patients.

In one embodiment, the invention provides a method of performing allele-specific gene silencing in mouse models or in human individuals affected by dominantly inherited CPVT, by administering to the subject in need thereof a vector carrying an expression cassette containing a promoter operably linked to sequences encoding a double stranded short interfering nucleic acid (siNA), wherein said siNA targets the RYR2 region containing the nucleotide mutation(s) and it is optimized to obtain a high knockdown rate of the mutant mRNA by sequence complementarity, leaving almost unaltered the expression of the wild type RYR2 transcript.

As used herein, siNA molecule denotes a short interfering RNA (siRNA), double-stranded RNA (dsRNA), micro-RNA (miRNA), short hairpin RNA (shRNA) or a circular RNA molecule.
The targeted RYR2 gene sequences may be murine-specific or human-specific. In human CPVT patients, the gene is the human RYR2 (NM_001035.2; coding sequence: SEQ ID NO:1).
In general, the alleles of the RYR2 gene will differ by one up to seven base pairs to be targeted by allele specific silencing.
In addition to using siNA molecules targeting the RYR2 regions, which contain the nucleotide change/s, the inventors have found that common SNPs can be exploited to generate interfering nucleic acids that selectively silence the mutant RYR2 expression. This alternative approach to RYR2 mutant allele-specific silencing is particularly convenient given the large number of different patient-specific disease causing mutations.

Common disease causing mutations in human RYR2 gene include, but are not limited to, R2474S, R4497C, R176Q, P2328S, Q4201R, V4653F, R176Q, T2504M, C2277R, E1724K, A2254V, A2394G, F4020L, E4076K, N4104I, H4108N, H4108Q, G4662S, H4762P, V4771I, P4902S, N4014K, and N4895D.

Common single nucleotide polymorphisms comprise most of the genetic diversity between humans and the RYR2 gene contains single nucleotide polymorphisms that can be separately targeted in one allele or in the other.

In another embodiment, the invention provides a method of performing allele-specific gene silencing in mouse models or in human individuals affected by dominantly inherited CPVT, by administering to the subject in need thereof a vector carrying an expression cassette containing a promoter operably linked to sequences encoding a double stranded short interfering nucleic acid (siNA), wherein said siNA targets common single nucleotide polymorphisms (SNPs) in the coding region of the RYR2 gene and said SNPs co-segregate with the mutations in the same allele or in the opposite, whereby the RYR2 allele in which the mutation is present is silenced, leaving almost unaltered the expression of the wild type RYR2 transcript.

SNPs typing and linkage analysis between the SNPs and the mutation may easily be assessed at the time of genetic screening that is routine in CPVT patients or at the time in which a patient has been advised to be treated with a gene therapy approach.

A bioinformatic assessment of the frequency and distribution of SNPs has been made using the available databases (Exome Variant Server) and a cohort of CPVT patients, to identify SNPs that have a minor allele frequency (MAF) between 30% and 40% and which thereby are relatively common but not too common to result in a high proportion of homozygous carriers of the minor allele, as of course they are not suitable to act as surrogate targets for the mutation as the same sequence at the SNPs site is present on both alleles.

By performing bioinformatics research, three main SNPs have been identified: rs3765097 (c.1359C>T; p.S453S), rs684923 (c.7806C>T; p.H2602H) and rs34967813 (c.8873A>G; p.Q2958R). The MAF for these SNPs according to different data bases is reported in Table 1.

TABLE 1
Information about three common SNPs in the RYR2 coding sequence taken from the Exome Variant Server. They are
mostly prevalent in heterozygosity so they represent potential valid targets for allele specific silencing.

| Variant | | | | | | | | |
|---|---|---|---|---|---|---|---|---|
| GRCh37 | | | MAF (%) | | mRNA | GVS | cDNA | Protein |
| Pos | rs ID | Alleles | (EA/AA/All) | Genes | Accession # | Function | Change | Change |
| 1:237617757 | rs3765097 | C > T | 39.949/ | RYR2 | NM_001035.2 | coding- | c.1359C > T | p.(S453=) |
| | | | 32.141/ | | | synonymous | | |
| | | | 48.8068 | | | | | |
| 1:237814783 | rs684923 | C > T | 43.6082/ | RYR2 | NM_001035.2 | coding- | c.7806C > T | p.(H2602=) |
| | | | 48.3406/ | | | synonymous | | |
| | | | 45.0733 | | | | | |
| 1:237841390 | rs34967813 | A > G | 30.4163/ | RYR2 | NM_001035.2 | missense | c.8873A > G | p.(Q2958R) |
| | | | 5.2618/ | | | | | |
| | | | 22.4871 | | | | | |

It has been estimated that using the three SNPs there would be 70% of heterozygous carriers with at least one of the three SNPs.

To test this estimate, we have performed a targeted analysis in a cohort of 176 patients, genotyped for RYR2 mutation-linked CPVT to quantify the percentage of carriers of these three SNPs. We observed that 138 individuals have at least one of the three variant in heterozygosity while only 38 patients have none of the polymorphisms in heterozygosity.

Therefore, by creating just six specific siNAs—e.g. miRNA—it would be possible to treat patients thereby enabling the allele specific silencing treatment for the vast majority of CPVT patients with RyR2 mutations.

Based on this approach, the following series of siRNA duplexes targeting the specific human nucleotide variants and its wild type counterpart have been designed. The 21-mer oligonucleotides are derived from siRNA duplex sequence that has demonstrated the best silencing potency and selectivity for the specific nucleotide change/s in the in vitro screening.

SNP: rs3765097 p. (S453S)

siRNA duplexes to test if causative mutation is in cis with rs3765097

(SEQ ID NO: 2)
AUUUGCCUAUAGAGUCCGUAAGCCUAAGUCUGCAGGAUCUCAU
UGGCUACUUC;
(SEQ ID NO: 3)
AUUUGCCUAUAGAGUCCGUAAGUCUAAGUCUGCAGGAUCUCAU
UGGCUACUUC;

TABLE 2

| | | 3′ |
|---|---|---|
| name | Seq 5′→3′ | overhang |
| siS453RYR2-U4 | AAGUCUAAGUCUGCAGGAUCU | -TT |
| | (SEQ ID NO: 4) | |
| siS453RYR2-U5 | UAAGUCUAAGUCUGCAGGAUC | -TT |
| | (SEQ ID NO: 5) | |
| siS453RYR2-U6 | GUAAGUCUAAGUCUGCAGGAU | -TT |
| | (SEQ ID NO: 6) | |
| siS453RYR2-U7 | CGUAAGUCUAAGUCUGCAGGA | -TT |
| | (SEQ ID NO: 7) | |
| siS453RYR2-U8 | CCGUAAGUCUAAGUCUGCAGG | -TT |
| | (SEQ ID NO: 8) | |
| siS453RYR2-U9 | UCCGUAAGUCUAAGUCUGCAG | -TT |
| | (SEQ ID NO: 9) | |
| siS453RYR2-U10 | GUCCGUAAGUCUAAGUCUGCA | -TT |
| | (SEQ ID NO: 10) | |
| siS453RYR2-U11 | AGUCCGUAAGUCUAAGUCUGC | -TT |
| | (SEQ ID NO: 11) | |
| siS453RYR2-U12 | GAGUCCGUAAGUCUAAGUCUG | -TT |
| | (SEQ ID NO: 12) | |
| siS453RYR2-U13 | AGAGUCCGUAAGUCUAAGUCU | -TT |
| | (SEQ ID NO: 13) | |
| siS453RYR2-U14 | UAGAGUCCGUAAGUCUAAGUC | -TT |
| | (SEQ ID NO: 14) | |
| siS453RYR2-U15 | AUAGAGUCCGUAAGUCUAAGU | -TT |
| | (SEQ ID NO: 15) | |
| siS453RYR2-U16 | UAUAGAGUCCGUAAGUCUAAG | -TT |
| | (SEQ ID NO: 16) | |
| siS453RYR2-U17 | CUAUAGAGUCCGUAAGUCUAA | -TT |
| | (SEQ ID NO: 17) | |
| siS453RYR2-U18 | CCUAUAGAGUCCGUAAGUCUA | -TT |
| | (SEQ ID NO: 18) | |

SiRNA duplexes to test if causative mutation is in trans with rs3765097

(SEQ ID NO: 19)
AUUUGCCUAUAGAGUCCGUAAGCCUAAGUCU
GCAGGAUCUCAUUGGCUACUUC;
(SEQ ID NO: 20)
AUUUGCCUAUAGAGUCCGUAAGUCUAAGUCU
GCAGGAUCUCAUUGGCUACUUC;

TABLE 3

| nome | Seq 5′→3′ | 3′overhang |
|---|---|---|
| siS453RYR2-C4 | AAGCCUAAGUCUGCAGGAUCU | -TT |
| | (SEQ ID NO: 21) | |
| siS453RYR2-C5 | UAAGCCUAAGUCUGCAGGAUC | -TT |
| | (SEQ ID NO: 22) | |
| siS453RYR2-C6 | GUAAGCCUAAGUCUGCAGGAU | -TT |
| | (SEQ ID NO: 23) | |
| siS453RYR2-C7 | CGUAAGCCUAAGUCUGCAGGA | -TT |
| | (SEQ ID NO: 24) | |
| siS453RYR2-C8 | CCGUAAGCCUAAGUCUGCAGG | -TT |
| | (SEQ ID NO: 25) | |
| siS453RYR2-C9 | UCCGUAAGCCUAAGUCUGCAG | -TT |
| | (SEQ ID NO: 26) | |
| siS453RYR2-C10 | GUCCGUAAGCCUAAGUCUGCA | -TT |
| | (SEQ ID NO: 27) | |
| siS453RYR2-C11 | AGUCCGUAAGCCUAAGUCUGC | -TT |
| | (SEQ ID NO: 28) | |
| siS453RYR2-C12 | GAGUCCGUAAGCCUAAGUCUG | -TT |
| | (SEQ ID NO: 29) | |
| siS453RYR2-C13 | AGAGUCCGUAAGCCUAAGUCU | -TT |
| | (SEQ ID NO: 30) | |
| siS453RYR2-C14 | UAGAGUCCGUAAGCCUAAGUC | -TT |
| | (SEQ ID NO: 31) | |
| siS453RYR2-C15 | AUAGAGUCCGUAAGCCUAAGU | -TT |
| | (SEQ ID NO: 32) | |
| siS453RYR2-C16 | UAUAGAGUCCGUAAGCCUAAG | -TT |
| | (SEQ ID NO: 33) | |
| siS453RYR2-C17 | CUAUAGAGUCCGUAAGCCUAA | -TT |
| | (SEQ ID NO: 34) | |
| siS453RYR2-C18 | CCUAUAGAGUCCGUAAGCCUA | -TT |
| | (SEQ ID NO: 35) | |

SNP: rs684923 p.(H2602H)

siRNA duplexes to test if causative mutation is in cis with rs684923

(SEQ ID NO: 36)
GAUGUUCCAUUAUUAAAUGAACACGCAAAGAUGCCUCUU
AAA;
(SEQ ID NO: 37)
GAUGUUCCAUUAUUAAAUGAACAUGCAAAGAUGCCUCUU
AAA;

TABLE 4

| Name | Seq 5′→3′ | 3′overhang |
|---|---|---|
| siH2602RYR2-U4 | ACAUGCAAAGAUGCCUCUUAA | -TT |
| | (SEQ ID NO: 38) | |
| siH2602RYR2-U5 | AACAUGCAAAGAUGCCUCUUA | -TT |
| | (SEQ ID NO: 39) | |
| siH2602RYR2-U6 | GAACAUGCAAAGAUGCCUCUU | -TT |
| | (SEQ ID NO: 40) | |
| siH2602RYR2-U7 | UGAACAUGCAAAGAUGCCUCU | -TT |
| | (SEQ ID NO: 41) | |
| siH2602RYR2-U8 | AUGAACAUGCAAAGAUGCCUC | -TT |
| | (SEQ ID NO: 42) | |
| siH2602RYR2-U9 | AAUGAACAUGCAAAGAUGCCU | -TT |
| | (SEQ ID NO: 43) | |
| siH2602RYR2-U10 | AAAUGAACAUGCAAAGAUGCC | -TT |
| | (SEQ ID NO: 44) | |
| siH2602RYR2-U11 | UAAAUGAACAUGCAAAGAUGC | -TT |
| | (SEQ ID NO: 45) | |
| siH2602RYR2-U12 | UUAAAUGAACAUGCAAAGAUG | -TT |
| | (SEQ ID NO: 46) | |
| siH2602RYR2-U13 | AUUAAAUGAACAUGCAAAGAU | -TT |
| | (SEQ ID NO: 47) | |
| siH2602RYR2-U14 | UAUUAAAUGAACAUGCAAAGA | -TT |
| | (SEQ ID NO: 48) | |
| siH2602RYR2-U15 | UUAUUAAAUGAACAUGCAAAG | -TT |
| | (SEQ ID NO: 49) | |
| siH2602RYR2-U16 | AUUAUUAAAUGAACAUGCAAA | -TT |
| | (SEQ ID NO: 50) | |
| siH2602RYR2-U17 | CAUUAUUAAAUGAACAUGCAA | -TT |
| | (SEQ ID NO: 51) | |
| siH2602RYR2-U18 | CCAUUAUUAAAUGAACAUGCA | -TT |
| | (SEQ ID NO: 52) | |

siRNA duplexes to test if causative mutation is in trans with rs684923

(SEQ ID NO: 53)
GAUGUUCCAUUAUUAAAUGAACACGCAAAGAUGCCUCUU
AAA;
(SEQ ID NO: 54)
GAUGUUCCAUUAUUAAAUGAACAUGCAAAGAUGCCUCUU
AAA;

TABLE 5

| Name | Seq 5′→3′ | 3′overhang |
|---|---|---|
| siH2602RYR2-4 | ACACGCAAAGAUGCCUCUUAA | -TT |
| | (SEQ ID NO: 55) | |
| siH2602RYR2-C5 | AACACGCAAAGAUGCCUCUUA | -TT |
| | (SEQ ID NO: 56) | |
| siH2602RYR2-C6 | GAACACGCAAAGAUGCCUCUU | -TT |
| | (SEQ ID NO: 57) | |
| siH2602RYR2-C7 | UGAACACGCAAAGAUGCCUCU | -TT |
| | (SEQ ID NO: 58) | |
| siH2602RYR2-C8 | AUGAACACGCAAAGAUGCCUC | -TT |
| | (SEQ ID NO: 59) | |
| siH2602RYR2-C9 | AAUGAACACGCAAAGAUGCCU | -TT |
| | (SEQ ID NO: 60) | |
| siH2602RYR2-C10 | AAAUGAACACGCAAAGAUGCC | -TT |
| | (SEQ ID NO: 61) | |
| siH2602RYR2-C11 | UAAAUGAACACGCAAAGAUGC | -TT |
| | (SEQ ID NO: 62) | |
| siH2602RYR2-C12 | UUAAAUGAACACGCAAAGAUG | -TT |
| | (SEQ ID NO: 63) | |
| siH2602RYR2-C13 | AUUAAAUGAACACGCAAAGAU | -TT |
| | (SEQ ID NO: 64) | |
| siH2602RYR2-C14 | UAUUAAAUGAACACGCAAAGA | -TT |
| | (SEQ ID NO: 65) | |
| siH2602RYR2-C15 | UUAUUAAAUGAACACGCAAAG | -TT |
| | (SEQ ID NO: 66) | |
| siH2602RYR2-C16 | AUUAUUAAAUGAACACGCAAA | -TT |
| | (SEQ ID NO: 67) | |
| siH2602RYR2-C17 | CAUUAUUAAAUGAACACGCAA | -TT |
| | (SEQ ID NO: 68) | |
| siH2602RYR2-C18 | CCAUUAUUAAAUGAACACGCA | -TT |
| | (SEQ ID NO: 69) | |

SNP: rs34967813 p.(Q2958R)

siRNA duplexes to test if causative mutation is in cis with rs34967813

(SEQ ID NO: 70)
GGAGAACAUUUCCCUUAUGAACAAGAAA
UCAAGUUCUUUGCAAAA;
(SEQ ID NO: 71)
GGAGAACAUUUCCCUUAUGAACGAGAAAU
CAAGUUCUUUGCAAAA;

TABLE 6

| Name | Seq 5′→3′ | 3′overhang |
|---|---|---|
| siQ2958R-RYR2- | AACGAGAAAUCAAGUUCUUUG | -TT |
| G4 | (SEQ ID NO: 72) | |
| siQ2958R-RYR2- | GAACGAGAAAUCAAGUUCUUU | -TT |
| G5 | (SEQ ID NO: 73) | |
| siQ2958R-RYR2- | UGAACGAGAAAUCAAGUUCUU | -TT |
| G6 | (SEQ ID NO: 74) | |
| siQ2958R-RYR2- | AUGAACGAGAAAUCAAGUUCU | -TT |
| G7 | (SEQ ID NO: 75) | |
| siQ2958R-RYR2- | UAUGAACGAGAAAUCAAGUUC | -TT |
| G8 | (SEQ ID NO: 76) | |
| siQ2958R-RYR2- | UUAUGAACGAGAAAUCAAGUU | -TT |
| G9 | (SEQ ID NO: 77) | |
| siQ2958R-RYR2- | CUUAUGAACGAGAAAUCAAGU | -TT |
| G10 | (SEQ ID NO: 78) | |
| siQ2958R-RYR2- | CCUUAUGAACGAGAAAUCAAG | -TT |
| G11 | (SEQ ID NO: 79) | |
| siQ2958R-RYR2- | CCCUUAUGAACGAGAAAUCAA | -TT |
| G12 | (SEQ ID NO: 80) | |
| siQ2958R-RYR2- | UCCCUUAUGAACGAGAAAUCA | -TT |
| G13 | (SEQ ID NO: 81) | |
| siQ2958R-RYR2- | UUCCCUUAUGAACGAGAAAUC | -TT |
| G14 | (SEQ ID NO: 82) | |
| siQ2958R-RYR2- | UUUCCCUUAUGAACGAGAAAU | -TT |
| G15 | (SEQ ID NO: 83) | |
| siQ2958R-RYR2- | AUUUCCCUUAUGAACGAGAAA | -TT |
| G16 | (SEQ ID NO: 84) | |
| siQ2958R-RYR2- | CAUUUCCCUUAUGAACGAGAA | -TT |
| G17 | (SEQ ID NO: 85) | |
| siQ2958R-RYR2- | ACAUUUCCCUUAUGAACGAGA | -TT |
| G18 | (SEQ ID NO: 86) | |

siRNA duplexes to test if causative mutation is in trans with rs34967813

(SEQ ID NO: 87)
GGAGAACAUUUCCCUUAUGAACAAGAAAUC
AAGUUCUUUGCAAAA;
(SEQ ID NO: 88)
GGAGAACAUUUCCCUUAUGAACGAGAAAUC
AAGUUCUUUGCAAAA;

TABLE 7

| Name | Seq 5′→3′ | 3′overhang |
|---|---|---|
| siQ2958R-RYR2- | AACAAGAAAUCAAGUUCUUUG | -TT |
| A4 | (SEQ ID NO: 89) | |
| siQ2958R-RYR2- | GAACAAGAAAUCAAGUUCUUU | -TT |
| A5 | (SEQ ID NO: 90) | |
| siQ2958R-RYR2- | UGAACAAGAAAUCAAGUUCUU | -TT |
| A6 | (SEQ ID NO: 91) | |
| siQ2958R-RYR2- | AUGAACAAGAAAUCAAGUUCU | -TT |
| A7 | (SEQ ID NO: 92) | |
| siQ2958R-RYR2- | UAUGAACAAGAAAUCAAGUUC | -TT |
| A8 | (SEQ ID NO: 93) | |
| siQ2958R-RYR2- | UUAUGAACAAGAAAUCAAGUU | -TT |
| A9 | (SEQ ID NO: 94) | |
| siQ2958R-RYR2- | CUUAUGAACAAGAAAUCAAGU | -TT |
| A10 | (SEQ ID NO: 95) | |
| siQ2958R-RYR2- | CCUUAUGAACAAGAAAUCAAG | -TT |
| A11 | (SEQ ID NO: 96) | |
| siQ2958R-RYR2- | CCCUUAUGAACAAGAAAUCAA | -TT |
| A12 | (SEQ ID NO: 97) | |
| siQ2958R-RYR2- | UCCCUUAUGAACAAGAAAUCA | -TT |
| A13 | (SEQ ID NO: 98) | |
| siQ2958R-RYR2- | UUCCCUUAUGAACAAGAAAUC | -TT |
| A14 | (SEQ ID NO: 99) | |
| siQ2958R-RYR2- | UUUCCCUUAUGAACAAGAAAU | -TT |
| A15 | (SEQ ID NO: 100) | |
| siQ2958R-RYR2- | AUUUCCCUUAUGAACAAGAAA | -TT |
| A16 | (SEQ ID NO: 101) | |
| siQ2958R-RYR2- | CAUUUCCCUUAUGAACAAGAA | -TT |
| A17 | (SEQ ID NO: 102) | |
| siQ2958R-RYR2- | ACAUUUCCCUUAUGAACAAGA | -TT |
| A18 | (SEQ ID NO: 103) | |
| siQ2958R-RYR2- | AACAUUUCCCUUAUGAACAAG | -TT |
| A19 | (SEQ ID NO: 104) | |

### BRIEF DESCRIPTION OF THE DRAWINGS

FIG. 1: flow chart depicting the steps that in the clinics will be used to choose the suitable siRNA to silence the allele containing the RyR2 mutation

FIG. 2: experimental protocol used to screen multiple siRNA duplexes in transient expression system using reporter alleles to simulate endogenous heterozygous expression of wild type and mutant RYR2 mRNA expression.

FIG. 3: Assessment of wild type (black) and mutant (white) allele expression by RealTime PCR in Hek293 cells transiently transfected with reporter alleles and siRNA duplexes

FIG. 4: Fluorescence analysis on in Hek293 cells transiently transfected with reporter alleles and siRNA duplexes

FIG. 5: Western Blot using specific antibody against HA (Wt allele) and FLAG (Mut allele) sequence in Hek293 cells transiently transfected with reporter alleles and siRNA duplexes

FIG. 6: miRYR2-U10 expression cassette was cloned into the pAAV2.1 adeno associated viral vector backbone plasmid. The resulting plasmid was used for the production of AAV9_miRyR2-U10 particles to infect RyR2 ^{R4496C/+} heterozygous mice in order to study in vivo the functional effects of the therapy.

FIG. 7: Assessment of wild type (black) and mutant (white) allele expression by RealTime PCR in Hek293 cells transiently transfected with reporter alleles and pAAV2.1-miRyRU10 or pAAV2.1-miRNAscramble.

FIG. 8: Western Blot using specific antibody against HA (WT allele) and FLAG (Mut allele) sequence in Hek293 cells transiently transfected with reporter alleles and pAAV2.1-miRyRU10 or pAAV2.1-miRNAscramble.

FIG. 9: Isolated cardiomyocytes (Phase Contrast, PhC) from infected animals were observed with fluorescence microscope in order to assess the presence and the level of expression of the reporter gene (EmGFP).

FIG. 10: Examples of triggered activity in isolated cardiomyocytes coming from negative GFP cells (not infected RYR2-R4496C^{+/-} cells) and positive GFP cells (infected RYR2-R4496C^{+/-} cells with AAV2/9-EmGFP-miRYR2)

FIG. 11: Evaluation of the incidence of ventricular arrhythmias following allele-specific silencing administration. A, In vivo Epinephrine and Caffeine administration elicited bidirectional ventricular tachycardia in Het and in Het-SCR, but not in Het-U10 mice. B, Quantification of the incidence of ventricular arrhythmias (VT) in Het, Het-SCR and Het-U10 mice infected at p8 (***P<0.001). C, Quantification of the incidence of ventricular arrhythmias (VT) in Het, Het-SCR and Het-U10 mice infected at p30 (*P<0.05; ***P<0.001).

FIG. 12: Electron Microscopy analysis of CRUs in WT and RyR2^{R4496C/+} mice treated with allele specific silencing. A, In WT cardiomyocytes the jSR cisternae are usually narrow and flat. Calsequestrin-2 (CASQ2) is clearly visible as a chain-like electron-dense line that runs parallel to the SR membrane (single black arrow). Smaller arrows in A point to the cytoplasmic domain, or feet, of RYR2s, spanning the narrow junctional gap between SR and plasmalemma. B, In Het cardiomyocytes the shape of jSR is more variable and slightly wider and do not always contain the chain-like electrondense polymer of CASQ2. C, In Het-SCR cardiomyocytes CRUs appear as in Het cardiomyocytes. D, Viral infection in Het-U10 rescues and restore the CRUs profile. Scale bar: 0.1 mm.

FIG. 13: Electron Microscopy analysis of contractile elements and mitochondria in WT and RyR2 R^{4496C/+} mice treated with allele specific silencing. A-E, Representative electron micrographs of cardiac cells in WT (A), Het (B-C), Het-SCR (D) Het-U10 (E). Insets show a detail of mitochondrial internal cristae. F, Quantitative analysis of the percentage of cardiac cells presenting severe structural abnormalities (Het, Het-SCR and Het-U10 vs. WT,*P<0.05; Het-U10 vs. Het, §P<0.05; Het-SCR vs. Het, #not significant). Scale bars=panels A-E, 1 μm; insets, 0.2 μm.

FIG. 14: Assessment of wild type (c.1359C) (black) and S453S SNP (c.1359T) containing allele (white) expression by RealTime PCR in Hek293 cells transiently transfected with hRYR2 reporter alleles and siRNA duplexes.

### CLINICAL APPROACH FOR THE APPLICATION OF ALLELE SPECIFIC SILENCING TARGETING SNPS TO SUPPRESS THE MUTANT TRANSCRIPT

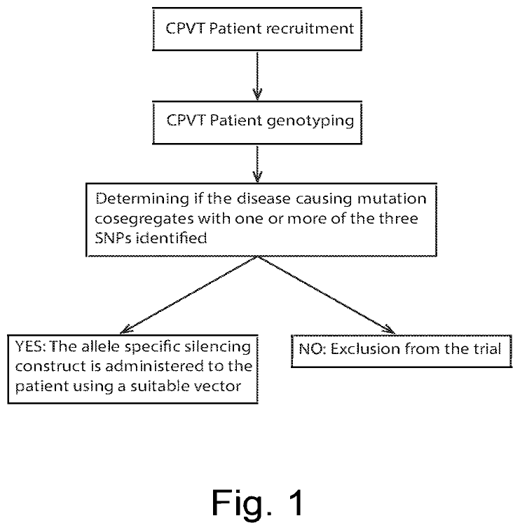

The flow chart depicting the steps that in the clinics will be used to choose the suitable siRNA to silence the allele containing the RYR2 mutation is shown in FIG. 1.

The therapy will be available in six different products to target the WT or the Mutant variant of each of the 3 SNPs, and siNAs will be developed to target the RNA regions containing the sequences of interest: 1359C; 1359T; 7806C; 7806T; 8873A; 8873G.

Each CPVT patient carrier of a pathogenic mutation in the RYR2 gene who is a candidate for gene therapy through allele specific silencing will be genotyped to determine the co-segregation of the disease causing mutation and the three SNPs.

Once the variant(s) that co-segregate with the mutation is (are) identified, the patient may be suitable to be treated with 1 or 2 or 3 products. The selection of the product to be used will be based on the sequence with the highest selectivity between mutant and WT allele.

In human patients the double-stranded short interfering nucleic acid is targeted to common SNPs including, but not limited to, rs3765097 (c.1359C>T), rs684923 (c.7806C>T) and rs34967813 (c.8873A>G), when they are in heterozygosity, so that they can be used to discriminate the allele carrying the disease causing mutation from the wild-type. This makes possible to generate few siNA sequences that can silence different patient-specific mutations in the RYR2 gene.

In a preferred aspect the engineered pre-siNA (e.g. pre-miRNA) expression cassette is inserted in a vector, preferably into a viral vector. The pre-siNA coding sequence is operably linked to a promoter, which could be CMV, or cardiac specific promoters such as: cTnT, TnC, α-MHC, MLC-2 and other tissue specific promoters.

The engineered pre-siNA expression cassette may be advantageously inserted in the serotype 9 adeno-associated viral (AAV2/9) vector. Alternatively, the engineered pre-siNA expression cassette may be advantageously inserted in the serotype 6 adeno-associated viral (AAV2/6) vector or serotype 8 adeno-associated viral (AAV2/8) vector.

Once the engineered pre-miRNA expression cassette is introduced into the cardiac cells for expression, the pre-miRNA forms an intramolecular stem loop structure similar to the structure of endogenous pre-miRNA that is then processed by the endogenous Dicer enzyme into a mature miRNA (Cullen et al., 2004).

The method according to the present invention allows the correction of the bidirectional and polymorphic arrhythmias in animal models with autosomal dominant CPVT by a viral gene therapy method by which mutant Ryanodine receptor type 2 mRNA is selectively knocked down by an artificially expressed miRNA.

Artificial miRNA expressing vector should be delivered preferably to the cardiac myocytes and expressed, whereby the normal and anti-arrhythmic contractile function of the heart is restored.

In another embodiment, the invention provides a method of in vitro screening of multiple allele-specific siRNA duplexes under heterozygous conditions, comprising co-transfection of two reporter alleles and siRNAs duplexes with known sequence into cultured HEK-293 cells and determining if the mutant allele is substantially silenced while the wild-type allele retains substantially normal expression.

Specifically, the invention provides a method for identifying an siNA capable of selectively silencing a mutant allele of the RYR2 gene compared to the wild-type allele of the RYR2 gene, comprising:
i. co-transfecting HEK-293 cells with mutant and wild-type reporter alleles and a multiplicity of siNA duplexes,
ii. determining if the mutant allele is substantially silenced relative to the wild-type allele, and
iii. determining the siNA associated with the substantial silencing;
thereby identifying the siNA capable of selectively silencing the mutant allele relative to the wild-type allele of the RYR2 gene.

In another preferred aspect, the siNA molecule according to the present invention advantageously allows to prevent or revert structural abnormalities of the CRUs and in the mitochondria that are associated with the R4496C mutation in the RyR2 gene.

### Experimental Setting

In this study, the gene is the murine RyR2 (NM_023868.2) and the targeted nucleotide variant is the C13483T on the protein coding mRNA leading to the R4496C amino acid change in the murine RyR2 protein.

Allele specific targeting study to silence the allele that includes the R4496C mutation in the RYR2 gene.

### AAV Mediated RNA Interference Approach to Induce Allele Specific Silencing of Mutant Gene in a RyR2 ^{R4496C/+} Mouse Model of Catecholaminergic Polymorphic Ventricular Tachycardia

1) Screening Multiple siRNAs in a Transient Expression System Using Reporter Alleles

Cellular models were used to test whether it is possible to target mutant allele in a transient expression system. We performed a series of in vitro mRNA and protein based assays to screen multiple potential siRNAs in order to identify siRNAs that would both recognize and efficiently silence the mutated allele preferentially over the wild-type allele.

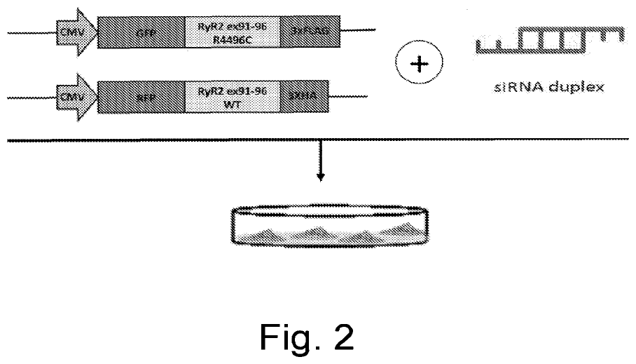

Using this system, the effects of a series of siRNA duplexes on mutant alleles in allele-specific silencing, as well as off-target silencing against WT alleles, can be examined under heterozygous conditions generated by co-transfecting two reporter alleles and siRNA duplexes into cultured HEK-293 cells (FIG. 2). As reporter alleles, two plasmids were generated containing:
1) CMV promoter followed by a reporter gene (Red Fluorescent Protein, RFP) in-frame linked with the murine cDNA sequence, corresponding to the WT-mRYR2 (exons 91 to 96), and to a tag sequence (3xHA) (FIG. 2).
2) CMV promoter followed by a reporter gene (Green Fluoresent

Protein, GFP) in-frame linked with the murine cDNA sequence, corresponding to the R4496C-mRYR2 (exons 91 to 96), and to a tag sequence (3xFLAG) (FIG. 2).

To induce such allele specific-RNAi, we designed siRNAs that carry nucleotide variations characterizing target disease allele in order to discriminate it from corresponding wild-type allele. Nucleotide sequences of wild-type and mutant RYR2 mRNAs and designed siRNAs are represented below (Table 8) and are based on the sequence of the 5→3′ sense-strand (passenger) siRNA element; mutant recognition site (MRS) is underlined (Table 8).

Wild Type RYR2 mRNA
(SEQ ID NO: 105)
5′-AACAGAAGCTGCTGAACTATTTTGCTCGCAA
CTTTTACAACATGAGAATGCTGGCC-3′
Mutant RYR2 mRNA
(SEQ ID NO: 106)
5′-AACAGAAGCTGCTGAACTATTTTGCTTGCAA
CTTTTACAACATGAGAATGCTGGCC-3′

TABLE 8
sequences of portion of the wild type and
mutant RYR2 cDNA and of the tested siRNA
duplexes

| name | Seq 5′->3′ | 3′overhang |
|---|---|---|
| SiRYR2-U5 | UGCUUGCAACUUUUACAACAU | -TT |
| | (SEQ ID NO: 107) | |
| SIRYR2-U6 | UUGCUUGCAACUUUUACAACA | -TT |
| | (SEQ ID NO: 108) | |
| SIRYR2-U7 | UUUGCUUGCAACUUUUACAAC | -TT |
| | (SEQ ID NO: 109) | |
| SiRYR2-U8 | UUUUGCUUGCAACUUUUACAA | -TT |
| | (SEQ ID NO: 110) | |
| SiRYR2-U9 | AUUUUGCUUGCAACUUUUACA | -TT |
| | (SEQ ID NO: 111) | |
| SiRYR2-U10 | UAUUUUGCUUGCAACUUUUAC | -TT |
| | (SEQ ID NO: 112) | |
| SIRYR2-U11 | CUAUUUUGCUUGCAACUUUUA | -TT |
| | (SEQ ID NO: 113) | |
| SIRYR2-U12 | ACUAUUUUGCUUGCAACUUUU | -TT |
| | (SEQ ID NO: 114) | |
| siRYR2-U13 | AACUAUUUUGCUUGCAACUUU | -TT |
| | (SEQ ID NO: 115) | |
| SIRYR2-U14 | GAACUAUUUUGCUUGCAACUU | -TT |
| | (SEQ ID NO: 116) | |
| siRYR2-U15 | UGAACUAUUUUGCUUGCAACU | -TT |
| | (SEQ ID NO: 117) | |
| SIRYR2-U16 | CUGAACUAUUUUGCUUGCAAC | -TT |
| | (SEQ ID NO: 118) | |
| SIRYR2-U17 | GCUGAACUAUUUUGCUUGCAA | -TT |
| | (SEQ ID NO: 119) | |

2) Assessment of Wild Type and Mutant Allele Expression by RealTime PCR, Fluorescence Microscopy and Western Blot in Transiently Transfected Hek293 Cells

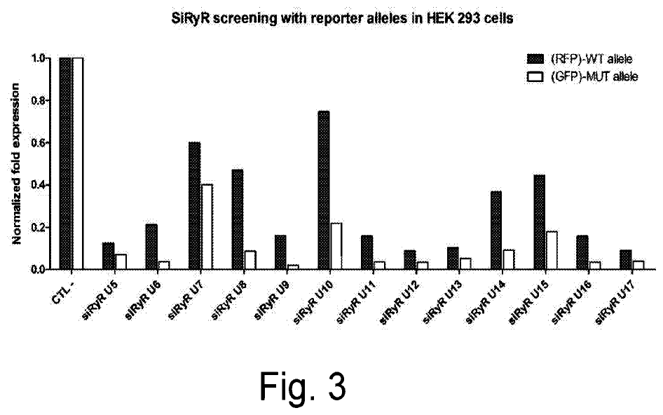

The effects of the designed siRNA duplexes on suppression of both the mutant and wild-type alleles have been subsequently examined by RealTime PCR, amplifying with specific primers GFP and RFP gene, to quantify the wild type and mutated allele mRNA respectively (Expression data have been analyzed using the 2^{-ΔΔCt} method, normalized on GAPDH expression and relative to the cells treated with scramble siRNA) (FIG. 3).

Most of siRNA duplexes have demonstrated a strong effect in suppressing RYR2 mRNA expression. Moreover, some of them were quite selective for the mutant allele.

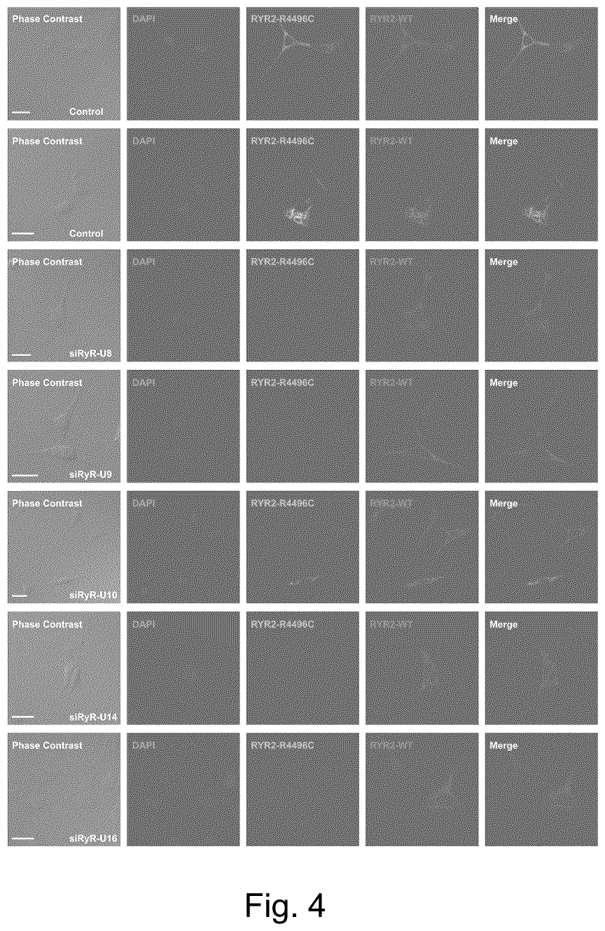

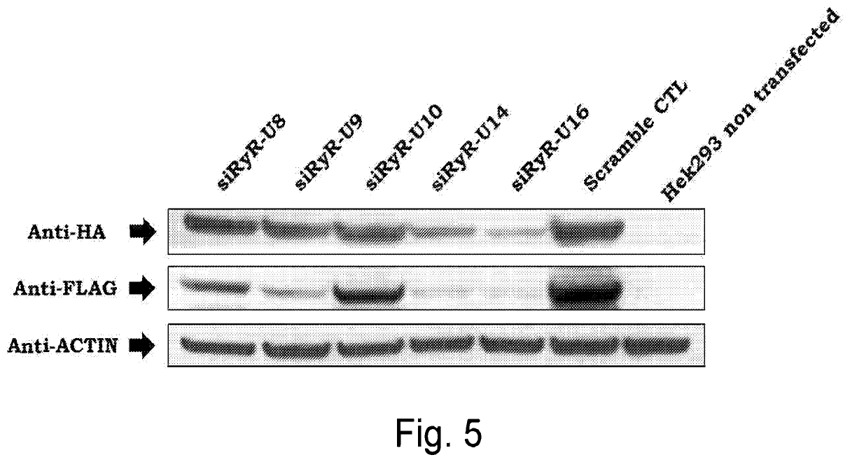

Therefore, we choose five siRNAs from this first screening (siRyR-U8, U9, U10, U14 and U16) and deeply analyzed their effect by confocal microscopy, to visualize green and red fluorescence (FIG. 4), and by Western Blot, using specific antibodies anti-HA and -FLAG epitope, to assess the relative protein expression of wild type and mutated allele respectively (FIG. 5).

3) Cloning and Validation of the Candidate siRNA into an Artificial miRNA-Expressing AAV Backbone Plasmid

From the previous step we selected siRyR-U10 as the candidate to be cloned into an artificial miRNA expression vector that allows the continuous and long term expression of the silencing molecule.

This siRNA was promising since it induces a weak suppression on the Wild Type allele but a strong silencing on the mutant one.

As an intermediate vector we used the BLOCK-iT™ Pol II miR RNAi Expression Vector (Life Technologies). This vector has a triple advantage over the conventionale Pol III-shRNA expression plasmids:
1. Polimerase II transcribed artificial miRNAs are expressed at tolerability levels while maintaining potent gene silencing capacities compared to shRNA, that can induce toxicity because of their unregulated and massive expression from Pol III promoters.
2. Co-cistronic expression of Emerald GFP (EmGFP), results in correlation of EmGFP expression with knockdown from our mi-RNAi.
3. Strong expression from the CMV immediate early promoter, with the option to use tissue-specific or other regulated promoters (or tissue specific).

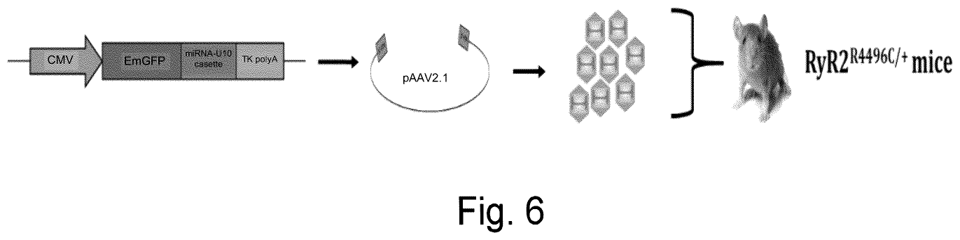

Subsequently, a fragment consisting in CMV promoter, EmGFP, pre-miRNA sequence and TKpolyA was amplified from the BLOCK-iT™ Pol II miR RNAi Expression Vector (Life Technologies) and sub-cloned into the adeno associated viral backbone vector pAAV2.1 provided by the Adeno-Associated Virus (AAV) vector Core facility (Tigem, Napoli, Italy) (FIG. 6).

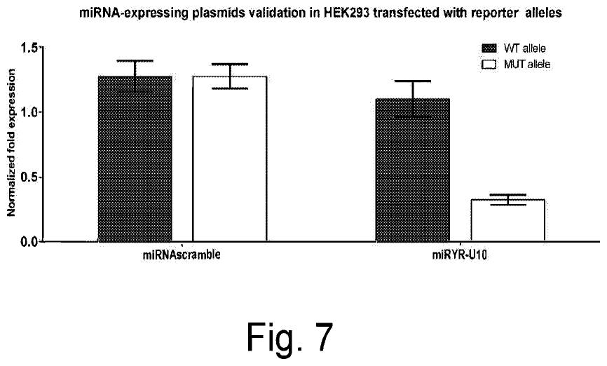

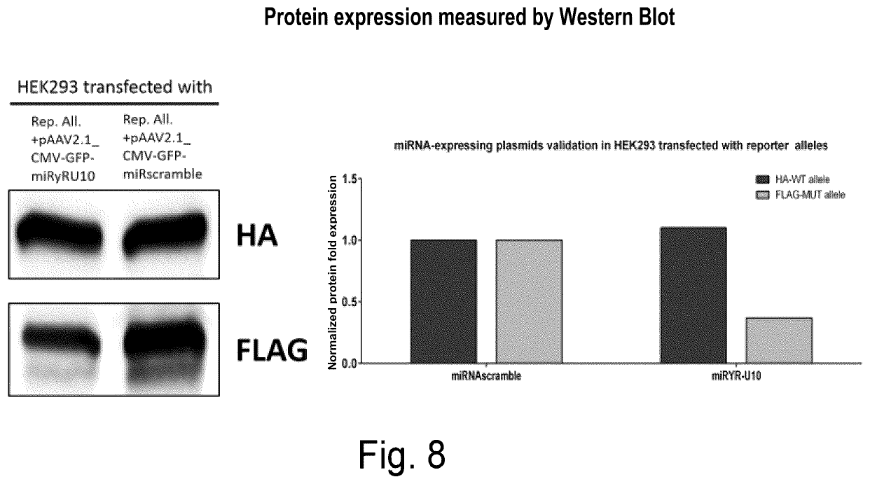

The resulting plasmid has been validated by RealTime (FIG. 7) and Western Blot (FIG. 8) analysis in the Hek293 cellular system, with heterozygous condition created through the transfection of the two reporter alleles. It was demonstrated that the miRYR2-U10 retains the capacity of siRYR-U10 in substantially suppressing mutant allele expression over the wild type. Expression data were compared to results obtained in cells transfected with reporter alleles and the miRNA-Scramble expressing plasmid (FIGS. 7-8).

4) In Vivo Infection of Cardiac Murine Myocytes Using the AAV2/9 Vector for Efficient miRYR2-U10 Transfer

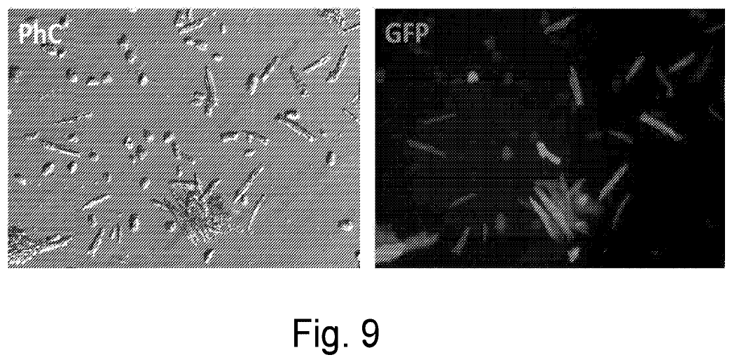

We infected, by intraperitoneal (I.P.) injection, neonates (P8/P9 after birth) RyR2 ^{R4496C/+} heterozygous mice using 100 μl of serotype 9 adeno-associated viral (AAV2/9) vector containing miRYR2-U10 expressing cassette (FIG. 5). The mice were monitored during their development and we did not observe any differences in comparison with the non-infected littermates. To evaluate the infection efficiency in the mice, we performed a standard procedure of cardiac myocytes isolation by enzymatic digestion 8 weeks after infection (4). The isolated cells were plated on coverslips and observed with fluorescence microscope in order to assess the presence and the level of expression of the reporter gene, eGFP (FIG. 9).

5) AAV2/9-miRYR2-U10 Infection Restores the Functional Phenotype of RyR2 ^{R4496C/+} Heterozygous Cardiac Cells

From our previous investigation we knew that CPVT arrhythmias are caused by delayed after depolarizations (DADs) and triggered activity (TA) at the level of a single cardiomyocyte. Using patch clamp techniques (in current clamp mode) we analyzed the development of the DADs and/or TA in basal condition and after adrenergic stimulation.

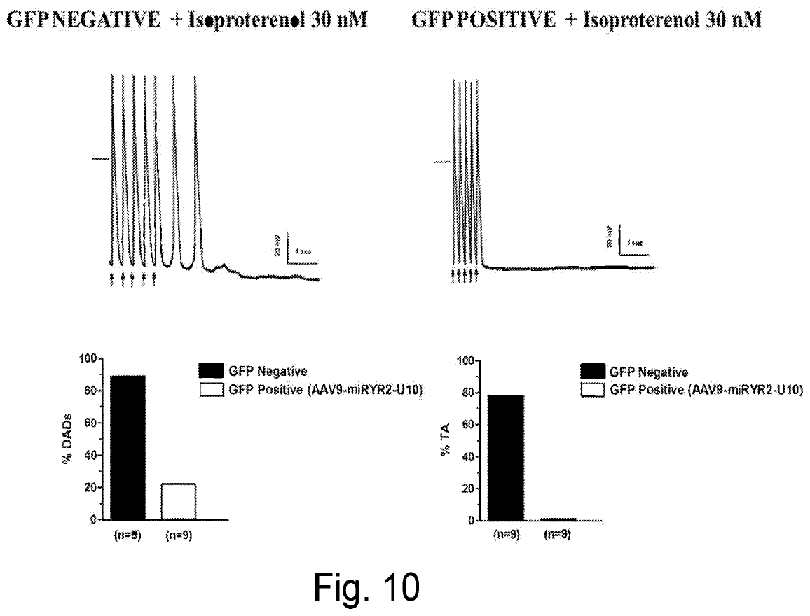

Epifluorescence signal (from the EmGFP present in our viral construct) was used to differentiate between non-infected (i.e. non-fluorescent) and infected (i.e. green fluorescent) cells and to perform comparative assay of DAD and TA occurrence. Isolated myocytes were paced at 5 Hz frequency at 1.5-fold the diastolic threshold and action potential was continuously recorded. An average of 67% of GFP negative (non-fluorescent) cells presented TA after ISO (30 nM) stimulation, while in the same experimental condition, only 6% of the GFP positive infected cells did (FIG. 10).

6) In Vivo Correction of the Dysfunctional Properties Observed in the RyR2 ^{R4496C/+} Mice

We used subcutaneous ECG telemeters to monitor and compare the incidence of arrhythmias in resting conditions and during adrenergic stress induced by epinephrine and caffeine injection.

We know from the previous characterization of our autosomal dominant CPVT mouse model that at least 50%-60% of RyR2 ^{R4496C/+} heterozygous mice present bidirectional ventricular tachycardia during adrenergic stress induced by epinephrine and caffeine injection (Cerrone M et al., 2005). Conversely, when we performed in vivo characterization of the arrhythmogenic substrate in our RyR2 ^{R4496C/+} heterozygous CPVT mouse model infected with AAV9-miRYR2-U10 we observed that on 10 treated mice only one developed ventricular arrhythmias (10%).

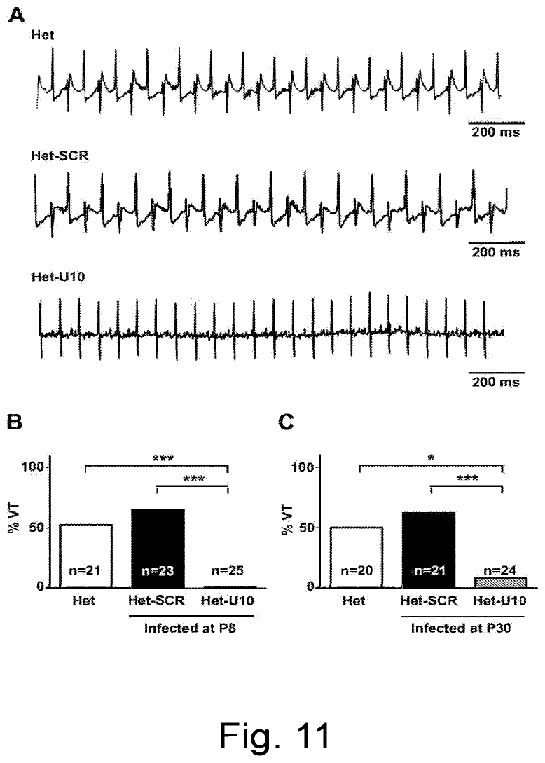

We performed experiments to assess whether administration of the therapeutic construct tested in neonatal mice would also be able to revert the arrhythmic substrate in adult mice. We therefore studied a new set of animals comparing arrhythmic events occurring in 8-week old RyR2 ^{R4496C/+} heterozygous mice (Het) versus those observed AAV9-miRYR2-U10 (Het-U10) and AAV9-miRNA-Scamble (Het-SCR) infected RyR2 ^{R4496C/+} heterozygous mice two months after infection (FIG. 11A). Data showed that 52% of Het mice (11/21) and 65% of Het-SCR (15/23) mice exhibited the typical bidirectional ventricular tachycardia, while treatment with miRYR2-U10 completely prevented the development of arrhythmias (0/25; Het-U10 vs Het-SCR ***P<0.001; Het-U10 vs Het ***P<0.001; FIG. 11B). In vivo evaluation of arrhythmias susceptibility was performed also in 3-months-old mice two months after viral delivery in adult age revealing a remarkable reduction of the ventricular tachycardia occurrence in Het-U10 (2/24, 8%) in comparison with the Het-SCR (13/21, 62%) and Het mice (10/20, 50%; Het-U10 vs Het-SCR ***P<0.001; Het-U10 vs Het *P<0.05; FIG. 11C). This set of data demonstrate that allele specific silencing-based gene therapy not only prevents occurrence of arrhythmic events when administered at birth but also reverts the arrhythmogenic substrate when delivered in post-puberal animals.

7) Morphological Alterations of CRUs in RYR2 ^{R4497C/WT} Hearts are Rescued by the AAV2/9-miRYR2-U10 Viral Infection.

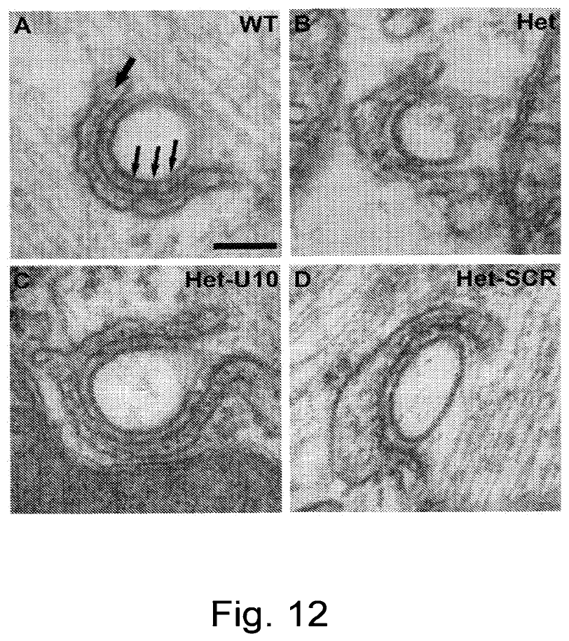

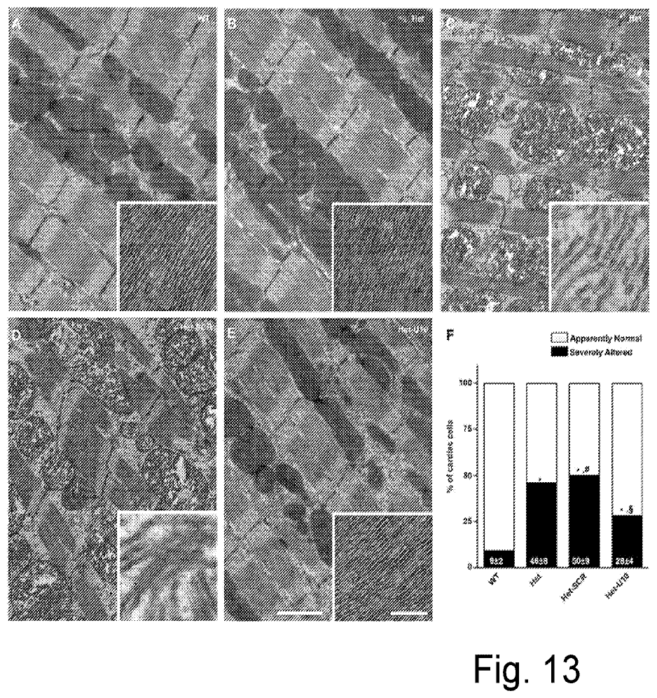

We performed electron microscopy on cardiac tissue of WT and RyR2 ^{R4496C/+} heterozygous mice to investigate whether in analogy with mice with recessive CPVT (Denegri M et al., 2014) also mice with the dominant form of CPVT present ultrastructural abnormalities and we observed abnormalities in the structure of the calcium release units (CRUs) (FIG. 12). On the surface of the jSR the Ryanodine Receptor channels can be visualized (FIG. 12A, small arrows). In WT cardiomyocytes the jSR cisternae are usually narrow and flat. Calsequestrin-2 (CASQ2) is clearly visible as a chain-like electron-dense line that runs parallel to the SR membrane (FIG. 12A). In RyR2 ^{R4496C/+} cardiomyocytes the shape of jSR is more variable and slightly wider and do not always contain the chain-like electrondense polymer of CASQ2 (FIG. 12; single black arrow). In cardiomyocytes from RyR2 R^{4496C/+} heterozygous mice infected with AAV2/9-miRNA-Scramble (Het-SCR) CRUs appear as in Het cardiomyocytes (FIG. 12D), while viral infection with AAV2/9-miRYR2-U10 rescues and restore the CRUs profile (Het-U10; FIG. 12C). Interestingly, we observed also that while cardiac samples from WT mice have contractile elements well aligned laterally with each other and mitochondria distributed longitudinally between myofibrils, that exhibit an electron dense matrix with parallel and tightly packed internal cristae (FIG. 13A), approximately 46% of myocytes from heart of Het mice presented damaged mitochondria with increased empty cytoplasmic spaces and alterations of the contractile elements (FIG. 13B-C). Of relevance hearts treated with AAV2/9-miRYR2-U10 (Het-U10; FIG. 13E), but not those treated with AAV2/9-miRNA-Scramble (Het-SCR; FIG. 13D), showed a reduction in the percentage of cardiac cells with severe mitochondrial abnormalities (from 46% in Het to 28% in Het-U10; FIG. 13F).

8) In Vitro Identification of Allele Specific Silencing Molecules Able to Suppress Expression of Transcripts Containing the rs3765097 (c.1359C>T; p.S4535) or its WT counterpart in the human RYR2 gene.

To transfer the method above described also to the human RYR2 gene and common SNPs that co-segregate with the mutations in the same allele or in the opposite, in a way that the hRYR2 allele in which the mutation is present is silenced, leaving almost unaltered the expression of the wild type RYR2 transcript, we performed a series of in vitro mRNA- and protein-based assays to screen multiple potential siRNAs in order to identify molecules that would both recognize and efficiently silence the SNP containing allele preferentially over the wild-type allele (mimicking the situation in which the SNP is in cis with the mutation) and viceversa (mimicking the situation in which the SNP is in trans with the mutation). The siRNA tested are sequences from SEQ ID NO:4 to SEQ ID NO:18 to target the T-containing allele and from SEQ ID NO:21 to SEQ ID NO:35 to target the C-containing allele.

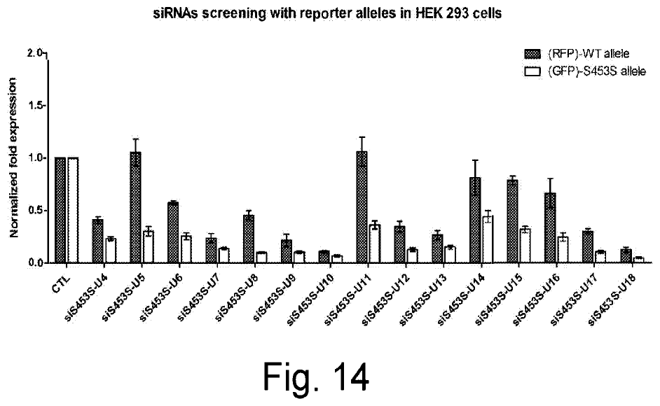

The effects of tested siRNA duplexes in allele-specific silencing, as well as off-target effects, have been examined under heterozygous conditions generated by co-transfecting two reporter alleles and siRNA duplexes into cultured HEK-293 cells. As reporter alleles, two plasmids were generated containing:
1) CMV promoter followed by a reporter gene (Red Fluorescent Protein, RFP) in-frame linked with the murine cDNA sequence, corresponding to the WT-hRYR2 (exons 11 to 15), and to a tag sequence (3xHA).
2) CMV promoter followed by a reporter gene (Green Fluoresent Protein, GFP) in-frame linked with the murine cDNA sequence, corresponding to the S453S-hRYR2 (exons 11 to 15), and to a tag sequence (3xFLAG).
   Of interest, several siRNAs targeted to rs3765097 in exon 15 of human RYR2 gene are able to suppress expression of the polymorphism carrier allele leaving minimally altered the expression of the non-carrier one (see FIG. 14).

### Materials and Methods

Animal Use

Animals were maintained and bred at the Charles River Laboratories in Calco, Italy, and transferred to the Maugeri Foundation for characterization of the phenotype. Animals were maintained and studied according to the protocols approved by the Animal Care and Use facility at the Maugeri Foundation. The adeno-associated virus delivery was via intra-caudal vein and/or intraperitoneal injection of 100-200 μl of purified virus in adult mice (8 weeks old) and/or neonatal mice (before the 9^{th} day after birth, P9) with a gauge syringe.

Quantitative Real-Time PCR

Real-time PCR was performed using the Bio-Rad CFX96 Real-Time PCR Detection System and analyzed using the Bio-Rad CFX Manager software package (Bio-Rad Laboratories, Inc., USA). Briefly, total RNA was purified with Rneasy mini kit (Qiagen) from Hek293 cells transiently transfected with reporter alleles and siRNA duplexes or with reporter alleles and pAAV2.1-miRyRU10 or pAAV2.1-miRNAscramble. Absorbance at 260 nm (A260) was measured for each RNA sample using the NanoDrop (ND-1000) spectrophotometer (NanoDrop Technologies, Wilmington, DE, USA). A total amount of 1 μg template RNA was used for retrotranscription performed with iScript cDNA Synthesis kit (Bio-Rad Laboratories, Inc., USA). Quantitative real-time PCR analysis was performed in optical 96-well plates using CFX96 detection module (Bio-Rad Laboratories, Inc.) in triplicate with SsoFast EvaGreen Supermix using specific primer mix to selectively amplify GFP or RFP sequence

(Forward:
(SEQ ID NO: 120)
5′-CTATATCATGGCCGACAAGCAG-3′,
(SEQ ID NO: 121)
5′-GCGTGATGAACTTCGAGGACG-3′
Reverse:
(SEQ ID NO: 122)
5′-GCTCGTCCATGCCGAGCGTG-3′,
(SEQ ID NO: 123)
5′-CAGCCCATGGTCTTCTTCTGC,
FLAG or HA
(Forward:
(SEQ ID NO: 124)
5′-GAACCTCCAGCGATACTGC-3′,
Reverse:
(SEQ ID NO: 125)
5′-CTGGTACCCTTGTCATCGTCATCCTTGTAATCG-3′,
(SEQ ID NO: 126)
5′ - CTGGTAACCTATTAAGCGTAGTCAGGTAC,

to quantify mutated allele or wild type mRNA respectively, and 20 ng of cDNA template. Values for threshold cycle (Ct) determination were generated automatically by the Bio-Rad CFX Manager software 1.5. GAPDH was used as internal reference using the following primers:

Forward:
(SEQ ID NO: 127)
5′-AAATCCCATCACCATCTTCC-3′
and
Reverse:
(SEQ ID NO: 128)
5′-GGTTCACACCCATGACGAAC-3′

Florescence Microscopy

Hek293 cells transiently transfected with reporter alleles and siRNA duplexes were fixed on coverslips in 3,7% paraformaldehyde for 10 minutes at room temperature. Coverslips were then washed in PBS with gentle shaking. The cells were washed several times in PBS and mounted on slides with mounting medium (Dako Fluorescent Mounting Medium, Dako North America, Inc, CA). Confocal microscopy was performed with a Leica TCS-SP2 digital scanning confocal microscope equipped with a HCX PL APO 40×/numerical aperture=1.25 oil immersion objective. We used the 488-nm Argon laser line for excitation of EmGFP and 594 nm He/Ne laser line for excitation of RFP. The pinhole diameter was kept at Airy 1. Images were exported to Adobe Photoshop CS3 (Adobe Systems, Mountain View, CA).

Immunoblotting

Hek293 cells transiently transfected with reporter alleles and siRNA duplexes or with reporter alleles and pAAV2.1-miRyRU10 or pAAV2.1-miRNAscramble have been lysated in RIPA buffer and total proteins extracted. Total proteins (30 μg/sample, quantified by the BCA assay) were resolved by SDS-gel electrophoresis, Mini PROTEAN TGX Stain-Free 4-15% gradient Gels (BIORAD) using 10× Tris/Glycine/SDS buffer (BIORAD), and blotted on 0,2 μm nitrocellulose using Trans Blot Turbo Transfer System (BIORAD). The membranes were probed with different antibodies: anti-FLAG (F3165, SIGMA), anti-HA (H3663, SIGMA) and anti-Actin (A1978, SIGMA) as reference protein. Secondary antibodies were conjugated with HRP (1:5000, Promega). Specific signals were developed using the Clarity Western ECL substrate (BIORAD) and detected using ChemiDoc MP Imaging System (BIORAD).

ECG Monitoring and Drug Testing

ECG radiotelemetry monitors (Data Sciences International) were implanted subcutaneously under general anaesthesia (Avertin 0.025 mg/kg). Body temperature was maintained at 37° C. by use of a thermally controlled heating pad (Harvard Apparatus). After 72 hours of recovery from surgery, phenotype characterization was performed. First, basal ECG was recorded for 10 minutes looking for the presence of arrhythmias. Subsequently, mice were injected with epinephrine and caffeine (2 and 120 mg/kg, respectively, by I.P.) to induce ventricular arrhythmias under a controlled stimulus. All animal were freely moving while ECG recordings were performed.

Isolation of Adult Mice Ventricular Myocytes

Ventricular myocytes were isolated using an established enzymatic digestion protocol (Hilal-Dandan et al., 2000) from RyR2 ^{R4496C/+} heterozygous mice, RyR2 ^{R4496C/+} heterozygous mice infected with AAV9-miRyR2-U10 and wild-type (WT) mice (8 weeks) of either sex.

Electrophysiological Recordings in Isolated Ventricular Myocytes

Cardiomyocytes were seeded on a glass bottom perfusion chamber mounted on the stage of an inverted microscope. After 5 minutes, the myocytes were bathed with the solution containing (in mmol/L): 140 NaCl, 4 KCl, 2 CaCl₂, 1 MgCl₂, 10 HEPES, and 5 glucose, pH 7.4, with NaOH. A thermostatically controlled heating ring surrounding the dish was used to maintain the bath solution at 35° C. Transmembrane potentials were recorded in whole cell current clamp mode using a MultiClamp 700B amplifier (Axon Instruments). Patch electrodes were pulled from borosilicate glass (WPI, Inc.) on a P-97 horizontal puller (Sutter Instruments). The electrodes had a resistance of 2 to 3 MΩ when filled with patch electrode solutions containing (in mmol/L): 120 potassium aspartate, 20 KCl, 1 MgCl₂, 4 Na₂ ATP, 0.1 GTP, 10 HEPES, 10 glucose, pH 7.2, with NaOH. All signals were acquired at 10 kHz (Digidata 1322A, Axon Instruments) and analyzed with the use of personal computer running pCLAMP version 9.2 software (Axon Instruments). Only quiescent, calcium-tolerant, rod-shaped cells with clear cross striations and a resting potential of less than or equal to −80 mV were used for electrophysiological recordings. Myocytes were electrically stimulated by intracellular current injection through patch electrodes using depolarizing pulses with duration of 3 ms and amplitude of 1.5 times the minimal current needed to evoke and action potential. The liquid junction potential between pipette and bath solution was calculated with pCLAMP software and corrected after experiments.

Vector Design and Production

The siRYR2-U10 siRNA duplex sequence, designed to target RYR2 mRNA (NM_023868.2) containing the R4496C mutation, was cloned into an artificial miRNA expression vector, BLOCK-iT™ Pol II miR RNAi Expression vector (Life Technologies, Cat. No: K4936-00), that allows continuous and long term expression of the silencing molecule. The cloning procedure was based on ligation of annealed oligonucleotides (5′TGCTGTAAAAGTTGCAAGCAAAATAGTTTTG 3′ (SEQ ID NO:129), 5′GCCACTGACTGACTATTTTGCGCAACTTTTAC 3′ (SEQ ID NO:130), 5′CCTGGTAAAAGTTGCGCAAAATAGTCAGTCA 3′ (SEQ ID NO:131), 5′GTGGCCAAAACTATTTTGCTTGCAACTTTTAC 3′ (SEQ ID NO:132) with the linearized vector (pcDNA™6.2-GW/EmGFPmiR-(Life Technologies, Cat. No: K4936-00)).

From the obtained plasmid, a fragment consisting in CMV promoter, EmGFP, premiRNA sequence and TKpolyA was amplified by PCR with specific primers (Forward: 5′ TAGCTAGCTGCTTCGCGATGTACGG 3′ (SEQ ID NO:133) and Reverse 5′ GTGAATTCGAACAAACGACCCAACACCCG 3′ (SEQ ID NO:134) including the NheI (Forward) and Eco RI (Reverse) cloning site and inserted into the pre-digested Nhe I-Eco RI sites adeno associated viral backbone vector pAAV-2.1 provided by the Adeno-Associated Virus (AAV) vector Core facility (Tigem, Napoli, Italy). All the used plasmids were sequenced.

The AAV production was done in collaboration with the Tigem core facility (http://www.tigem.it/core-facilities/adeno-associated-virus-aav-vector-core). The AAV vectors were produced using a transient transfection of 3 plasmids in 293 cells: pAd helper, pAAV rep-cap (packaging), pAAV Cis (including our insert, miRYR2, cloned in the pAAV2.1-CMV-eGFP plasmid MCS). The vectors were purified by CsCl centrifugation and undergo quality control such as Real Time PCR and Dot Blot analysis for physical titer, or Comassie staining of SDS PAGE to evaluate the presence and purity of capsid proteins, the infectivity (eGFP⁺ cells/ml, only for CMV-eGFP preps) and the sterility (for preps to be used in large animals). The service returned with a viral preparation in PBS with a total yield>1×10¹² genome copies. All AAV stocks were frozen at −80° C. in single vial and thawed during the surgical procedure.

Electron Microscopy

Hearts isolated from WT, heterozygous RyR2 ^{R4496C/+} and infected heterozygous RyR2^{R4496C/+} mice, were fixed by retrograde aortic perfusion with 3.5% glutaraldehyde in 0.1 mol/L NaCaCo buffer (pH 7.2) and analyzed. Small bundles of papillary muscles were post-fixed in 2% OsO₄ in NaCaCo buffer for 2 hours and then block-stained in saturated uranyl acetate. After dehydration, specimens were embedded in an epoxy resin (Epon 812). Ultrathin sections were cut in a Leica Ultracut R microtome (Leica Micro system, Austria) using a Diatome diamond knife (Diatome Ltd. CH-2501 Biel, Switzerland) and double stained with uranyl acetate and lead citrate. All sections were examined with an FP 505 Morgagni Series 268D electron microscope (FEI Company, Brno, Czech Republic), equipped with Megaview III digital camera and Soft Imaging System (Munster, Germany). The percentage of cardiac cells exhibiting severe structural alterations was quantified. Cells considered severely damaged are characterized by severe structural abnormalities affecting mitochondria in the majority of the interior. In most cases cardiac cells with severely altered mitochondria also present large area of apparently empty cytoplasmic spaces and alterations affecting contractile elements.

### Abbreviations

The following abbreviations have been used in the present specification: CASQ2, calsequestrin 2; CPVT, Catecholaminergic Polymorphic Ventricular Tachycardia; CICR, Calcium Induced Calcium Release; CRU, calcium release unit; DAD, Delayed afterdepolarization; EC coupling, excitation-contraction coupling; ECG, electrocardiogram; CMV, Citomegalovirus; GFP, green fluorescent protein; RFP, red fluorescent protein; AAV, Adeno Associated Virus; EP, electrophysiology; I.P., intraperitoneal; ISO, isoproterenol; RYR2, ryanodine receptor type 2; WT, Wild type; siRNA, small interfering RNA; miRNA, microRNA; SNP, Single Nucleotide Polimorphisms; HA, Human influenza hemagglutinin; MRS, Mutant Recognition Site; RNAi, RNA interference; TK polyA, HSV thymidine kinase (TK) polyadenylation signal sequence.

### REFERENCES

1. Priori S G, Napolitano C, Colombo B, Memmi M, Bloise R. Mutations of the cardiac Ryanodine receptor (RYR2) gene are associated to heterogeneous clinical phenotypes and high lethality. Circulation 2001; 104(suppl II):335.
2. Lahat H, Pras E, Olender T, Avidan N, Ben Asher E, Man O, Levy-Nissenbaum E, Khoury A, Lorber A, Goldman B, Lancet D, Eldar M. A missense mutation in a highly conserved region of CASQ2 is associated with autosomal recessive catecholamine-induced polymorphic ventricular tachycardia in Bedouin families from Israel. Am J Hum. Genet. 2001;69:1378-1384.
3. Bers, D. M. Cardiac excitation—contraction coupling. Nature 415, 198-205 (2002).
4. Franzini-Armstrong, C., Protasi, F. & Tijskens, P. The assembly of calcium release units in cardiac muscle. Ann. NY Acad. Sci. 1047, 76-85 (2005).
5. Pieske, B., Maier, L. S., Bers, D. M. & Hassenfuss, G. Ca²⁺ handling and sarcoplasmic reticulum Ca²⁺ content in isolated failing and nonfailing human myocardium. Circ. Res. 85, 38-46 (1999).
6. Venetucci L, Denegri M, Napolitano C, Priori S G. Inherited calcium channelopathies in the pathophysiology of arrhythmias. Nat Rev Cardiol. 9(10), 561-75 (2012).
7. Liu N, Colombi B, Memmi M, Zissimopoulos S, Rizzi N, Negri S, Imbriani M, Napolitano C, Lai F A, Priori S G. Arrhythmogenesis in Catecholaminergic Polymorphic Ventricular Tachycardia. Insights From a RyR2 R4496C Knock-In Mouse Model. Circulation Research 2006; 99:292-298.
8. Cerrone M, Colombi B, Santoro M, Raffale di Barletta M, Scelsi M, Villani L, Napolitano C, Priori S G. Bidirectional Ventricular Tachycardia and Fibrillation Elicited in a Knock-In Mouse Model Carrier of a Mutation in the Cardiac Ryanodine Receptor (RyR2). Circulation Research 2005; 96:e77-e82.
9. Denegri M, Bongianino R, Lodola F, Boncompagni S, DeGiusti V C, Avelino-Cruz J E, Liu N, Persampieri S, Curcio A, Esposito F, Pietrangelo L, Marty I, Villani L, Moyaho A, Baiardi P, Auricchio A, Protasi F, Napolitano C, Priori S G. A single delivery of an adeno-associated construct to transfer casq2 gene to knock-in mice affected by catecholaminergic polymorphic ventricular tachycardia is able to cure the disease from birth to advanced age Circulation. 2014; 129(25):267381
10. Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836):494-8
11. Hilal-Dandan, R., Kanter, J. R. & Brunton, L. L. Characterization of G-protein signaling in ventricular myocytes from the adult mouse heart: differences from the rat. J. Mol. Cell. Cardiol. 32, 1211-1221 (2000).

1.-30. (canceled)

31. A method of performing allele-specific gene silencing in a mammal affected by dominantly inherited catecholaminergic polymorphic ventricular tachycardia (CPVT), the method comprising administering to the mammal a vector encoding one or more short interfering nucleic acid (siNA) sequences, wherein the one or more siNA sequences target RNA associated with the expression of at least one mutation present in a mutant ryanodine receptor type 2 (RYR2) allele of the mammal, thereby silencing the mutant allele of RYR2 present in the mammal.

32. A method of performing allele-specific gene silencing in a mammal affected by dominantly inherited CPVT, the method comprising administering to the mammal a vector encoding one or more siNA sequences, wherein the one or more siNA sequences target RNA associated with the expression of one or more single nucleotide polymorphisms (SNPs) in the coding region of a mutant RYR2 gene, wherein said SNPs co-segregate with the RYR2 mutations in the same allele or in the opposite, whereby the RYR2 allele that carries the mutation is silenced.

33. The method according to claim 31, wherein said siNA is a double-stranded siNA molecule that inhibits expression of a mutant allele of the RYR2 gene.

34. The method according to claim 33, wherein said siNA comprises a sense strand and an antisense strand, and wherein the sense strand is complementary to the antisense strand.

35. The method according to claim 31, wherein the siNA molecule is a short interfering RNA (siRNA), a double-stranded RNA (dsRNA), a micro-RNA (miRNA), a short hairpin RNA (shRNA), or a circular RNA molecule.

36. The method according to claim 31, wherein the siNA molecule comprises a sense strand comprising a nucleotide sequence selected from the group consisting of SEQ ID NOs: 4 through 18; SEQ ID NOs: 21 through 35; SEQ ID NOs: 38 through 52; SEQ ID NOs: 55 through 69; SEQ ID NOs: 72 through 86; SEQ ID NOs: 89 through 104; and SEQ ID NOs: 107 through 119.

37. The method according to claim 31, wherein the antisense strand comprises a sequence that is complementary to at least a part of an RNA associated with the expression of the mutant allele.

38. The method according to claim 31, wherein the mutant allele of the RYR2 gene differs from wild-type RYR2 gene by up to seven nucleic acid residues.

39. The method according to claim 31, wherein the vector is a recombinant viral vector.

40. The method according to claim 39, wherein the vector is a serotype 9 adeno-associated viral (AAV2/9) vector, a serotype 6 adeno-associated viral (AAV2/6) vector, or a serotype 8 adeno-associated viral (AAV2/8) vector.

41. The method according to claim 32, wherein said siNA is a double-stranded siNA molecule that inhibits expression of a mutant allele of the RYR2 gene.

42. The method according to claim 41, wherein said siNA comprises a sense strand and an antisense strand, and wherein the sense strand is complementary to the antisense strand.

43. The method according to claim 32, wherein the siNA molecule is an siRNA, a dsRNA, a miRNA, an shRNA, or a circular RNA molecule.

44. The method according to claim 32, wherein the siNA molecule comprises a sense strand comprising a nucleotide sequence selected from the group consisting of SEQ ID NOs: 4 through 18; SEQ ID NOs: 21 through 35; SEQ ID NOs: 38 through 52; SEQ ID NOs: 55 through 69; SEQ ID NOs: 72 through 86; SEQ ID NOs: 89 through 104; and SEQ ID NOs: 107 through 119.

45. The method according to claim 32, wherein the antisense strand comprises a sequence that is complementary to at least a part of an RNA associated with the expression of the mutant allele.

46. The method according to claim 32, wherein the mutant allele of the RYR2 gene differs from wild-type RYR2 gene by up to seven nucleic acid residues.

47. The method according to claim 32, wherein the vector is a recombinant viral vector.

48. The method according to claim 47, wherein the vector is a serotype 9 adeno-associated viral (AAV2/9) vector, a serotype 6 adeno-associated viral (AAV2/6) vector, or a serotype 8 adeno-associated viral (AAV2/8) vector.

49. A method for identifying a siNA capable of selectively silencing a mutant allele of the RYR2 gene compared to the wild-type allele of the RYR2 gene, comprising:
(i) co-transfecting HEK-293 cells with mutant and wild-type reporter alleles and a multiplicity of siNA duplexes;
(ii) determining if the mutant allele is substantially silenced relative to the wild-type allele; and
(iii) determining the siNA associated with the substantial silencing;
thereby identifying the siNA capable of selectively silencing the mutant allele relative to the wild-type allele of the RYR2 gene.


SEQUENCE LISTING (134 sequences)

SEQ ID NO: 1 - DNA, 14904 bp, Homo sapiens
  source 1..14904: mol_type = genomic DNA
atggccgatgggggcgagggcgaagacgagatccagttcctgcgaactgatgatgaagtggttctgcagtgcaccgcaaccatccacaaagaacaacagaagctatgcttggcagcagaaggatttggcaacagactttgtttcttggagtccacttccaattccaagaatgtgcccccagacctctccatctgcacctttgtgctggagcagtccctctctgtccgggcgctgcaggagatgctggctaacaccgtggagaaatcagaagggcaagttgatgtggaaaaatggaaattcatgatgaagactgctcaaggtggtggtcatcgaacactcctctacggacatgccatattgctgcgccattcctatagtggcatgtatctgtgctgcctgtccacctcccggtcttcaactgataagctggcttttgatgttggcttgcaagaggacaccacaggggaggcttgttggtggaccatacaccctgcctctaagcagcgatcagaaggagaaaaagtacgagttggagatgacctcatcttagttagcgtgtcctctgaaaggtacttgcacttgtcttatggcaacggcagcttacacgtggatgccgctttccagcagactctctggagcgtggccccaatcagctcaggaagtgaggcagcccaagggtatctcattggtggtgatgtcctcaggttgctgcatggacacatggacgagtgtctcactgtcccttcaggagaacatggtgaagagcagcggagaactgttcattatgaaggtggcgctgtgtctgttcatgcacgttccctttggagactagagacgctaagagttgcgtggagtggaagccacataagatggggacagccattccgactacgccatgtcacaacaggaaaatacttgagtctcatggaagacaaaaaccttctactcatggacaaagagaaagctgatgtaaaatcaacagcatttaccttccggtcttccaaggaaaaattggatgtaggggtgagaaaagaagtagatggcatgggaacatctgaaataaaatacggtgactcagtatgctatatacaacatgtagacacaggcctatggcttacttaccagtctgtggacgtgaaatccgtgagaatgggatctatacaacgtaaggctattatgcatcatgaaggccacatggatgatggcataagtttgtcgagatcccagcatgaagaatcacgcacagcccgagttatccggagcacagtcttccttttcaatagatttataaggggccttgatgctctcagcaagaaagcgaaggcttccacagtcgatttgcctatagagtccgtaagcctaagtctgcaggatctcattggctacttccaccccccagatgagcatttagagcatgaagacaaacagaacagactacgagccctgaagaatcggcaaaatctcttccaggaagagggaatgatcaacctcgtgcttgagtgcatagaccgtttgcacgtctacagcagtgcagcacactttgctgatgttgctgggcgagaagcaggagagtcttggaaatccattctgaattctctgtatgagttgctggcggctctaattagaggaaatcgtaaaaactgtgctcaattttctggctccctcgactggttgatcagcagattggaaagactggaagcttcttcaggcattctggaagttttacactgtgttttagtagaaagtccagaagctctaaatattattaaagaaggacatattaaatctattatctcacttttagacaaacatggaagaaatcacaaggttctggatgtcttgtgctcactctgtgtttgccacggggttgcagtccgttctaaccagcatctcatctgtgacaatctcctaccaggaagagacttgttattgcagacacgtcttgtgaaccatgtcagcagcatgagacccaatatttttctgggcgtcagtgaaggttctgctcagtataagaaatggtactatgaattgatggtggaccacacagagccctttgtgacagctgaagcaactcacctgcgagtgggctgggcttccactgaaggatattctccctaccctggagggggcgaagagtggggtggaaatggtgttggagatgatctcttctcctatggatttgatggccttcatctctggtcaggttgtattgctcgtactgtaagctcaccaaaccaacatctgttaagaactgatgatgtcatcagttgctgtttagatctgagtgccccaagcatctcgttccgaattaatggacaacctgttcaaggaatgtttgagaatttcaacatcgatggcctcttctttccagtcgttagtttctctgcaggaataaaagtacgctttctgcttggagggcgacatggagaattcaaatttcttcctccacctgggtatgctccttgttatgaagctgttctgccaaaagaaaagttgaaagtggaacacagccgagagtacaagcaagaaagaacttacacacgcgacctgctgggccccacagtttccctgacgcaagctgccttcacacccatccctgtggataccagccagatcgtgttgcctcctcatctagaaagaataagagaaaaactggcagagaatatccatgaactctgggttatgaataaaattgagcttggctggcagtatggtccggttagagatgacaacaagagacaacacccatgcctggtggagttctccaagctgcctgaacaggagcgcaattacaacttacaaatgtcgcttgagaccctgaagactttgttggcattaggatgtcatgtgggtatatcagatgaacatgctgaagacaaggtgaaaaaaatgaagctacccaagaattaccagctgacaagtggatacaagcctgcccctatggacctgagctttatcaaactcaccccatcacaagaagcaatggtggacaagttggcagaaaatgcacataatgtgtgggcgcgggatcgaatccggcagggctggacttatggcatccaacaggacgtaaagaacagaagaaatcctcgccttgttccctacactcttctggatgaccgaaccaagaaatccaacaaggacagcctccgcgaggctgtgcgcacgctgctggggtacggctacaacttggaagcaccagatcaagatcatgcagccagagccgaagtgtgcagcggcaccggggaaaggttccgaatcttccgtgccgagaagacctatgcagtgaaggccggacggtggtattttgaatttgagacggtcactgctggagacatgagggttggttggagtcgtcctggttgtcaaccggatcaggagcttggctcagatgaacgtgcctttgcctttgatggcttcaaggcccagcggtggcatcagggcaatgaacactatgggcgctcttggcaagcaggcgatgtcgtggggtgtatggttgacatgaacgaacacaccatgatgttcacactgaatggtgaaatccttcttgatgattcaggctcagaactggctttcaaggactttgatgttggcgatggattcatacctgtgtgtagccttggagtggctcaagtgggtaggatgaactttggaaaggatgtcagcaccttgaaatatttcaccatctgtggcttacaagagggctatgaaccatttgccgttaatacaaacagggatattaccatgtggctgagcaagaggcttcctcagtttcttcaagttccatcaaaccatgaacatatagaggtgaccagaatagacggcaccatagacagttccccatgtttaaaggtcactcagaagtcttttggttctcagaacagcaacactgatatcatgttttatcgcctgagcatgccgatcgagtgcgcggaggtcttctccaagacggtggctggagggctccctggggctggcctttttgggcccaagaatgacttggaagattatgatgctgattctgactttgaggttctgatgaagacagctcatggccatctagtgcccgatcgtgttgacaaagacaaagaagctactaaaccagagtttaacaaccacaaagattatgcccaggaaaagccctctcgtctgaaacaaagatttttgcttagaagaacaaagccagattacagcacaagccattctgcaagactcaccgaagatgtccttgctgatgatcgggatgactatgatttcttgatgcaaacgtccacgtactattactcagtgagaatctttcctggacaagaacctgctaatgtctgggtgggctggattacatcagatttccatcagtatgacacaggctttgacttggacagagttcgcacagtaacagttactctaggagatgaaaaaggaaaagtgcatgaaagcatcaaacgcagcaactgctatatggtatgtgcgggtgagagcatgagccccgggcaaggacgcaacaataatggactggagattggctgtgtggtggatgctgccagcgggctgctcacattcattgccaatggcaaggaactgagcacatactatcaggtggaaccgagtacaaaattatttcctgcggtttttgcacaagctacaagtcccaatgttttccagtttgagttgggaagaataaagaatgtgatgcctctctcggcgggattattcaagagtgagcacaagaaccccgtgccgcagtgccccccgcgcctccacgtgcagttcctgtcacacgtcctgtggagcagaatgcccaaccagtttttgaaggtagatgtgtctcgaataagtgaacgccaaggctggttggtgcagtgtttggatcctctgcagttcatgtctcttcatatccctgaggaaaacagatctgttgacatcttagagttgacagagcaggaggaattgctgaaatttcactatcacactctccggctctactcagccgtctgtgctcttgggaaccaccgggtggcccatgccctgtgcagccatgtggatgaacctcagctcctctatgccattgagaacaagtacatgcctggtttgctgcgtgctggctactatgacctgctgattgacatccacctgagctcctatgccactgccaggctcatgatgaacaacgagtacattgtccccatgacggaggagacgaagagcatcaccctgttccctgatgagaacaaaaaacacggccttccagggatcggcctcagcacctccctcaggccacggatgcagttttcctcccccagttttgtaagcattagtaatgaatgttaccagtacagtccagagttcccactggacatcctcaagtccaaaaccatacagatgctgacagaagctgttaaagagggcagtcttcatgcccgggacccagttggagggactactgaattcctctttgtacctctcatcaagcttttctataccctgctgatcatgggcatctttcacaacgaggacttgaagcacatcttgcagttgattgagcccagtgtgtttaaagaagctgccactccggaggaggagagtgacacgctggagaaagagctcagtgtggacgatgcaaagctgcaaggagctggtgaggaagaagccaaggggggcaagcggcccaaggaaggcctgctccaaatgaaactgccagagccagttaaattgcagatgtgcctactgcttcagtacctctgtgactgccaggtccggcaccggatagaagccattgtagccttttcagatgattttgtggctaagctccaagacaatcaacgtttccgatacaacgaagtcatgcaagccttaaacatgtcagctgcactcacagccaggaagacaaaggaatttagatcaccacctcaagaacagatcaatatgcttctcaattttaaggatgacaaaagtgaatgtccatgtccagaagaaattcgtgaccaactattggatttccatgaagatttgatgacacattgtggaattgagctggatgaagatgggtctctggatggaaacagtgatttaacaattagagggcgtctgctatccctggtagaaaaggtgacatatctgaagaagaagcaagcagaaaaaccagttgagagtgactccaaaaagtcctccactctgcagcagctgatttctgagaccatggtccgatgggctcaggagtctgtcattgaagaccccgagctggtgagggccatgtttgtgttgctccatcggcagtatgacggcattgggggtcttgttcgggccctgccaaagacctacacgataaatggtgtgtccgtggaggacaccatcaacctgctggcatcccttggtcagattcggtccctgctgagtgtgagaatgggcaaagaagaagagaagctcatgattcgtggattaggggatattatgaataacaaagtgttttaccagcaccctaatctcatgagggcactggggatgcacgagactgtgatggaggtcatggtgaacgtccttggaggtggagagtccaaggaaatcacctttcccaagatggtggccaactgttgccgttttctctgttacttctgtcgtataagtaggcagaatcaaaaagctatgtttgatcatctcagttatttactggaaaacagcagtgttggtcttgcctccccagctatgagaggttcaacaccactggatgtggctgcagcttcggtgatggataataatgaactagcattagctctgcgtgagccggatctagaaaaggtagttcgttatttggctggttgtggactgcaaagttgccagatgctggtgtctaagggctatccagacattgggtggaacccagttgaaggagagagatatcttgactttcttagatttgctgtcttctgtaatggggagagtgtggaggaaaatgcaaatgtcgtggtgagattgctcattcggaggcctgagtgttttggtcctgctttgagaggagaaggtgggaatgggcttcttgcagcaatggaagaagccatcaaaatcgccgaggatccttcccgagatggtccctcaccaaatagcggatccagtaaaacacttgacacagaggaggaggaagatgacactatccacatggggaacgcgatcatgaccttctattcagctttgattgacctcttgggacgctgtgctcctgagatgcatttgattcatgccgggaagggagaagccatcagaattaggtccattttgagatccctcattcccctgggagatttggtgggcgttatcagcatcgcttttcagatgccaacaatagccaaagatgggaatgtggtggaacctgacatgtctgcggggttttgcccagatcacaaggcagccatggttttattccttgacagggtctatgggattgaggttcaagacttcctcctccatcttcttgaggttggctttctgccagatctccgggcggctgcttctttagatacggcagctttgagtgctacagacatggccttggccctcaatcggtacctttgcacagccgtcttgccattgttaacaagatgtgctcctctctttgctggcacagagcaccacgcttctctcattgactcattacttcatactgtgtatagactttctaagggctgttcacttaccaaagctcagcgggattccatagaagtttgtttactctctatttgtggacaactgagaccttctatgatgcagcacttactcagaagattagtatttgatgttccattattaaatgaacacgcaaagatgcctcttaaactgctgacaaatcattatgaaagatgctggaaatattactgcctgcctggagggtggggaaactttggtgctgcctcagaagaagaacttcatttatcaagaaagttgttctggggcatttttgatgccctgtctcaaaagaaatatgaacaagaacttttcaaactggcactgccttgcctgagtgcagttgcgggagctttgcctccagactacatggagtcaaattatgtcagtatgatggaaaaacagtcatcaatggattctgaagggaactttaacccacaacctgttgatacctcaaatattacaattcctgagaaattggaatacttcattaacaaatatgcagaacactcccatgacaaatggtcaatggacaagttggcaaatggatggatttatggagaaatatattcagactcttctaaggttcagccattaatgaagccatataagctattgtctgaaaaggaaaaagaaatttatcgctggccaatcaaagaatctttaaaaactatgctggcttggggctggagaattgaaagaactcgggagggagacagcatggccctttacaaccggactcgtcgtatttctcagacaagccaggtttctgtggacgctgcccatggttacagtccccgggccattgacatgagcaatgttacactatctagagacctgcatgctatggcagaaatgatggctgaaaactaccataatatatgggcaaagaaaaagaaaatggagttggagtccaaaggaggaggaaaccatcctctgctggtgccctatgatacactgacagccaaagagaaagccaaggatagagaaaaagcacaggacatcctcaagttcttgcagatcaatggatatgctgtatccagaggatttaaggacctggaactggacacgccttctattgagaaacgatttgcctatagtttcctccaacaactcattcgctatgtggatgaagcccatcagtatatcctggagtttgatggtggcagcagaggcaaaggagaacatttcccttatgaacaagaaatcaagttctttgcaaaagtcgttcttcctttaattgatcagtatttcaaaaaccatcgtttatacttcttatctgcagcaagcagacctctctgctctggaggacatgcttccaacaaagagaaagaaatggtgactagcctattctgcaaacttggagttcttgtcaggcataggatttcactatttggcaatgatgcaacatcaattgtcaactgtcttcatattttgggtcagactttggatgcaaggacagtgatgaagactggcctggagagtgttaaaagtgcactcagagcttttctggacaacgctgcagaggatctggagaagaccatggaaaacctcaagcagggccagttcactcacacccgaaaccagcccaaaggggttactcagattatcaattacaccacagtggccctgctgccaatgctgtcttcattatttgaacatattggccagcatcagttcggagaagacctaatattggaagatgtccaggtgtcttgttatagaattctgactagcttatatgctttgggaaccagcaagagtatttacgtggagaggcaacgttctgcattaggagaatgtctagctgcctttgctggtgcttttcctgtagcatttttggaaactcatctggacaaacataatatttactccatctacaataccaagtcttcacgagaaagagcagctctcagtttgccaactaatgtggaagatgtttgtccaaacataccgtctttggagaaactcatggaagaaatcgtggaattagccgagtccggcattcgctacactcaaatgccacatgtcatggaagtcatactgcccatgctttgcagctacatgtctcgttggtgggagcatggacctgagaacaatccagaacgggccgagatgtgctgcacagccctgaactcagagcacatgaacacacttctagggaacatattgaaaatcatatataataacttggggattgatgagggagcctggatgaagaggctagcagtgttttcccagcctataataaataaagtgaaacctcagctcttgaaaactcatttcttgccgttaatggagaaactcaagaaaaaggcagctacggtggtgtctgaggaagaccacctgaaagctgaggccaggggggacatgtcggaggcagaactcctcatcctagatgagttcaccacactggccagagatctctatgccttctaccctctcttgattagatttgtggactataacagggcaaagtggctaaaggagcctaacccagaagcagaggagctcttccgcatggtggctgaagtgtttatctactggtcgaagtcccataatttcaaaagagaagagcagaacttcgttgtacagaatgaaatcaacaatatgtctttccttattactgataccaagtcaaagatgtcaaaggcagctgtttctgatcaggaaaggaagaaaatgaagcgcaaaggagatcggtattccatgcagacctctctgattgtagcagctctgaagcggttactgcccattgggttgaacatctgtgcccctggggaccaggagctcattgctctggccaaaaatcgatttagcctgaaagataccgaggatgaagtacgagatataatccgcagcaatattcatttacaaggcaagttggaggatcctgctattagatggcaaatggctctttacaaagacttaccaaacaggactgatgatacctcagatccagagaagacggtagaaagagtattggatatagcaaatgtgctttttcatcttgaacagaagtctaaacgtgtgggtcggagacattactgtctggtggaacatcctcagagatctaaaaaggctgtatggcataaactactgtccaagcagaggaaaagggctgttgtagcctgcttccggatggcccccttatataatctgccaaggcatcgggctgtcaatctctttcttcagggatatgaaaagtcttggattgaaacagaagaacattactttgaagataaactgatagaagatttagcaaaacctggggctgaacctccagaagaagatgaaggcactaagagagttgatcctctacatcagctgatccttctgtttagtcggacagctttaacagagaaatgcaaactggaggaagattttttatatatggcctatgcagatattatggcaaagagttgtcatgatgaggaagatgacgatggtgaagaggaagtgaagagttttgaagaaaaagaaatggaaaagcaaaagcttctataccagcaagcccgactccacgatcgtggcgcggctgagatggtgctacagacaatcagtgccagcaaaggtgaaactggaccaatggtagcagctactctgaaacttggaattgctattttaaatggtgggaactccacagtacagcagaaaatgcttgactacctcaaggagaaaaaggatgtgggcttctttcagagcctggccggcctgatgcagtcatgtagtgtccttgacctaaatgcatttgagcgacaaaacaaagctgaaggtcttgggatggtgacagaggaaggatcaggagaaaaggttctgcaggacgatgagttcacctgtgacctcttccgattcctgcaactactctgtgagggacacaactcagattttcagaattatctgagaactcagactggcaataatacaactgtcaacataattatctccactgtagactacctactgagagttcaggaatcaattagtgacttttattggtattactctgggaaagatgttattgatgaacaaggacaacggaatttctccaaagctatccaagtggcaaaacaagtctttaacactcttacagagtatattcagggtccttgcactgggaatcaacagagtttggcacacagcaggctgtgggatgctgtggtcggctttcttcatgtgtttgcccatatgcagatgaagctgtcgcaggattccagtcaaattgagctattaaaagaattaatggatctgcagaaggatatggtggtcatgttgctgtccatgttagaaggtaatgttgttaatggaacgattggcaaacagatggtggatatgcttgtggaatcttccaacaacgtggagatgattctcaaattttttgacatgttcttaaaactaaaggatttgacgtcgtctgatacttttaaagaatatgaccccgatggcaagggagtcatttccaagagggacttccacaaagcgatggagagccataagcactacacgcagtcagaaacggaatttcttttgtcttgtgcggagacggatgagaatgaaaccctcgactacgaagagttcgtcaaacgcttccacgaacctgcgaaggacatcggcttcaacgtcgccgtccttctgacaaacctctctgagcacatgcccaacgatacccgacttcagacttttctggaattagcagagagcgtcctgaattatttccagccctttctgggccgcatcgaaatcatgggaagcgccaaacgcatcgagagggtctattttgaaatcagtgagtccagccgaacccagtgggagaagccccaggtcaaggagtccaaaagacagttcatatttgacgtggtcaacgaaggcggagagaaagagaagatggaactctttgtgaacttctgcgaggacaccatctttgaaatgcagctggcggctcagatctcggagtcggacttgaacgagaggtcagcgaataaggaagaaagcgagaaggagaggccggaagagcaggggccgaggatggctttcttctccattctgacggtcaggtcggccctgtttgcgctcaggtacaatatcttgacccttatgcgaatgctcagtctgaagagcctgaagaagcagatgaaaaaagtaaaaaagatgaccgtgaaggacatggtcacggccttcttttcatcctactggagtattttcatgaccctcttgcacttcgtggccagcgttttcagaggctttttccgcatcatttgcagcctgctgcttgggggaagcctcgtcgaaggtgctaaaaagatcaaagttgcagaactgttagccaacatgccagaccccactcaggatgaggttagaggagatggggaggagggagagaggaaacccctggaagccgccctgccctccgaggatctgaccgacttaaaggagctgacagaggaaagtgaccttctttcggacatctttggcctggatctgaagagagaaggaggacagtacaaactgattcctcataatccaaatgctgggctcagtgacctcatgagcaacccagtccccatgcctgaggtgcaggaaaaatttcaggaacagaaggcaaaagaagaagaaaaggaagaaaaagaagaaaccaaatctgaacctgaaaaagccgagggagaagatggagaaaaagaagagaaagccaaggaagacaagggcaaacaaaagttgaggcagcttcacacacacagatacggagaaccagaagtgccagagtcagcattctggaagaaaatcatagcatatcaacagaaacttctaaactattttgctcgcaacttttacaacatgagaatgttagccttatttgtcgcatttgctatcaatttcatcttgctcttttataaggtctccacttcttctgtggttgaaggaaaggagctccccacgagaagttcaagtgaaaatgccaaagtgacaagcctggacagcagctcccatagaatcatcgcagttcactatgtactagaggagagcagcggctacatggagcccacgttgcgtatcttagctattctgcacacggtcatttctttcttctgcatcattggatactactgcttgaaagtcccattggttatttttaagcgagaaaaggaagtggcacggaaattggaatttgatgggctttatattacagaacagccttcagaagatgatattaaaggccagtgggatagactcgtaatcaacacacagtcatttcccaacaactactgggacaaatttgttaaaagaaaggttatggataaatatggagagttctacggccgagacagaatcagtgaattacttggcatggacaaggcagctctggacttcagtgatgccagagaaaagaagaagccaaagaaagacagctccttatcagctgtactgaactccattgatgtgaagtatcagatgtggaaactaggagtcgttttcactgacaactccttcctctacctagcctggtatatgactatgtctgttcttggacactataacaactttttttttgccgctcaccttctcgacattgctatgggattcaagacattaagaaccatcttgtcctcagtaactcacaatggcaaacagctcgtattaaccgttggcttattagctgttgttgtatacctatacactgtggtggcattcaattttttccgaaaattctacaataaaagtgaagatggtgatacaccagatatgaaatgtgacgatatgctaacatgctatatgttccacatgtatgttggagttcgtgctggaggagggatcggggatgaaatcgaagacccagcaggagatgaatatgagatctatcgaatcatctttgacatcactttcttcttctttgttattgtcattctcttggccataatacaaggtctaattattgatgcttttggagaactaagagaccaacaggaacaagtcaaagaagacatggagaccaaatgcttcatctgtgggataggcaatgattacttcgacacagtgccacatggctttgaaacccacactttacaggagcacaacttggctaattacttgttttttctgatgtatcttataaacaaagatgaaacagaacacacaggacaggaatcttatgtctggaagatgtatcaagaaaggtgttgggaatttttcccagcaggggattgcttccggaaacagtatgaagaccagctaaattaa

SEQ ID NO: 2 - RNA, 53 nt, Homo sapiens
  source 1..53: mol_type = mRNA
atttgcctatagagtccgtaagcctaagtctgcaggatctcattggctacttc

SEQ ID NO: 3 - RNA, 53 nt, Homo sapiens
  source 1..53: mol_type = mRNA
atttgcctatagagtccgtaagtctaagtctgcaggatctcattggctacttc

SEQ ID NO: 4 - RNA, 21 nt, Homo sapiens
  source 1..21: mol_type = other RNA
aagtctaagtctgcaggatct

SEQ ID NO: 5 - RNA, 21 nt, Homo sapiens
  source 1..21: mol_type = other RNA
taagtctaagtctgcaggatc

SEQ ID NO: 6 - RNA, 21 nt, Homo sapiens
  source 1..21: mol_type = other RNA
gtaagtctaagtctgcaggat

SEQ ID NO: 7 - RNA, 21 nt, Homo sapiens
  source 1..21: mol_type = other RNA
cgtaagtctaagtctgcagga

SEQ ID NO: 8 - RNA, 21 nt, Homo sapiens
  source 1..21: mol_type = other RNA
ccgtaagtctaagtctgcagg

SEQ ID NO: 9 - RNA, 21 nt, Homo sapiens
  source 1..21: mol_type = other RNA
tccgtaagtctaagtctgcag

SEQ ID NO: 10 - RNA, 21 nt, Homo sapiens
  source 1..21: mol_type = other RNA
gtccgtaagtctaagtctgca

SEQ ID NO: 11 - RNA, 21 nt, Homo sapiens
  source 1..21: mol_type = other RNA
agtccgtaagtctaagtctgc

SEQ ID NO: 12 - RNA, 21 nt, Homo sapiens
  source 1..21: mol_type = other RNA
gagtccgtaagtctaagtctg

SEQ ID NO: 13 - RNA, 21 nt, Homo sapiens
  source 1..21: mol_type = other RNA
agagtccgtaagtctaagtct

SEQ ID NO: 14 - RNA, 21 nt, Homo sapiens
  source 1..21: mol_type = other RNA
tagagtccgtaagtctaagtc

SEQ ID NO: 15 - RNA, 21 nt, Homo sapiens
  source 1..21: mol_type = other RNA
atagagtccgtaagtctaagt

SEQ ID NO: 16 - RNA, 21 nt, Homo sapiens
  source 1..21: mol_type = other RNA
tatagagtccgtaagtctaag

SEQ ID NO: 17 - RNA, 21 nt, Homo sapiens
  source 1..21: mol_type = other RNA
ctatagagtccgtaagtctaa

SEQ ID NO: 18 - RNA, 21 nt, Homo sapiens
  source 1..21: mol_type = other RNA
cctatagagtccgtaagtcta

SEQ ID NO: 19 - RNA, 53 nt, Homo sapiens; identical to SEQ ID NO: 2
  source 1..53: mol_type = mRNA

SEQ ID NO: 20 - RNA, 53 nt, Homo sapiens; identical to SEQ ID NO: 3
  source 1..53: mol_type = mRNA

SEQ ID NO: 21 - RNA, 21 nt, Homo sapiens
  source 1..21: mol_type = other RNA
aagcctaagtctgcaggatct

SEQ ID NO: 22 - RNA, 21 nt, Homo sapiens
  source 1..21: mol_type = other RNA
taagcctaagtctgcaggatc

SEQ ID NO: 23 - RNA, 21 nt, Homo sapiens
  source 1..21: mol_type = other RNA
gtaagcctaagtctgcaggat

SEQ ID NO: 24 - RNA, 21 nt, Homo sapiens
  source 1..21: mol_type = other RNA
cgtaagcctaagtctgcagga

SEQ ID NO: 25 - RNA, 21 nt, Homo sapiens
  source 1..21: mol_type = other RNA
ccgtaagcctaagtctgcagg

SEQ ID NO: 26 - RNA, 21 nt, Homo sapiens
  source 1..21: mol_type = other RNA
tccgtaagcctaagtctgcag

SEQ ID NO: 27 - RNA, 21 nt, Homo sapiens
  source 1..21: mol_type = other RNA
gtccgtaagcctaagtctgca

SEQ ID NO: 28 - RNA, 21 nt, Homo sapiens
  source 1..21: mol_type = other RNA
agtccgtaagcctaagtctgc

SEQ ID NO: 29 - RNA, 21 nt, Homo sapiens
  source 1..21: mol_type = other RNA
gagtccgtaagcctaagtctg

SEQ ID NO: 30 - RNA, 21 nt, Homo sapiens
  source 1..21: mol_type = other RNA
agagtccgtaagcctaagtct

SEQ ID NO: 31 - RNA, 21 nt, Homo sapiens
  source 1..21: mol_type = other RNA
tagagtccgtaagcctaagtc

SEQ ID NO: 32 - RNA, 21 nt, Homo sapiens
  source 1..21: mol_type = other RNA
atagagtccgtaagcctaagt

SEQ ID NO: 33 - RNA, 21 nt, Homo sapiens
  source 1..21: mol_type = other RNA
tatagagtccgtaagcctaag

SEQ ID NO: 34 - RNA, 21 nt, Homo sapiens
  source 1..21: mol_type = other RNA
ctatagagtccgtaagcctaa

SEQ ID NO: 35 - RNA, 21 nt, Homo sapiens
  source 1..21: mol_type = other RNA
cctatagagtccgtaagccta

SEQ ID NO: 36 - RNA, 42 nt, Homo sapiens
  source 1..42: mol_type = mRNA
gatgttccattattaaatgaacacgcaaagatgcctcttaaa

SEQ ID NO: 37 - RNA, 42 nt, Homo sapiens
  source 1..42: mol_type = mRNA
gatgttccattattaaatgaacatgcaaagatgcctcttaaa

SEQ ID NO: 38 - RNA, 21 nt, Homo sapiens
  source 1..21: mol_type = other RNA
acatgcaaagatgcctcttaa

SEQ ID NO: 39 - RNA, 21 nt, Homo sapiens
  source 1..21: mol_type = other RNA
aacatgcaaagatgcctctta

SEQ ID NO: 40 - RNA, 21 nt, Homo sapiens
  source 1..21: mol_type = other RNA
gaacatgcaaagatgcctctt

SEQ ID NO: 41 - RNA, 21 nt, Homo sapiens
  source 1..21: mol_type = other RNA
tgaacatgcaaagatgcctct

SEQ ID NO: 42 - RNA, 21 nt, Homo sapiens
  source 1..21: mol_type = other RNA
atgaacatgcaaagatgcctc

SEQ ID NO: 43 - RNA, 21 nt, Homo sapiens
  source 1..21: mol_type = other RNA
aatgaacatgcaaagatgcct

SEQ ID NO: 44 - RNA, 21 nt, Homo sapiens
  source 1..21: mol_type = other RNA
aaatgaacatgcaaagatgcc

SEQ ID NO: 45 - RNA, 21 nt, Homo sapiens
  source 1..21: mol_type = other RNA
taaatgaacatgcaaagatgc

SEQ ID NO: 46 - RNA, 21 nt, Homo sapiens
  source 1..21: mol_type = other RNA
ttaaatgaacatgcaaagatg

SEQ ID NO: 47 - RNA, 21 nt, Homo sapiens
  source 1..21: mol_type = other RNA
attaaatgaacatgcaaagat

SEQ ID NO: 48 - RNA, 21 nt, Homo sapiens
  source 1..21: mol_type = other RNA
tattaaatgaacatgcaaaga

SEQ ID NO: 49 - RNA, 21 nt, Homo sapiens
  source 1..21: mol_type = other RNA
ttattaaatgaacatgcaaag

SEQ ID NO: 50 - RNA, 21 nt, Homo sapiens
  source 1..21: mol_type = other RNA
attattaaatgaacatgcaaa

SEQ ID NO: 51 - RNA, 21 nt, Homo sapiens
  source 1..21: mol_type = other RNA
cattattaaatgaacatgcaa

SEQ ID NO: 52 - RNA, 21 nt, Homo sapiens
  source 1..21: mol_type = other RNA
ccattattaaatgaacatgca

SEQ ID NO: 53 - RNA, 42 nt, Homo sapiens; identical to SEQ ID NO: 36
  source 1..42: mol_type = mRNA

SEQ ID NO: 54 - RNA, 42 nt, Homo sapiens; identical to SEQ ID NO: 37
  source 1..42: mol_type = mRNA

SEQ ID NO: 55 - RNA, 21 nt, Homo sapiens
  source 1..21: mol_type = other RNA
acacgcaaagatgcctcttaa

SEQ ID NO: 56 - RNA, 21 nt, Homo sapiens
  source 1..21: mol_type = other RNA
aacacgcaaagatgcctctta

SEQ ID NO: 57 - RNA, 21 nt, Homo sapiens
  source 1..21: mol_type = other RNA
gaacacgcaaagatgcctctt

SEQ ID NO: 58 - RNA, 21 nt, Homo sapiens
  source 1..21: mol_type = other RNA
tgaacacgcaaagatgcctct

SEQ ID NO: 59 - RNA, 21 nt, Homo sapiens
  source 1..21: mol_type = other RNA
atgaacacgcaaagatgcctc

SEQ ID NO: 60 - RNA, 21 nt, Homo sapiens
  source 1..21: mol_type = other RNA
aatgaacacgcaaagatgcct

SEQ ID NO: 61 - RNA, 21 nt, Homo sapiens
  source 1..21: mol_type = other RNA
aaatgaacacgcaaagatgcc

SEQ ID NO: 62 - RNA, 21 nt, Homo sapiens
  source 1..21: mol_type = other RNA
taaatgaacacgcaaagatgc

SEQ ID NO: 63 - RNA, 21 nt, Homo sapiens
  source 1..21: mol_type = other RNA
ttaaatgaacacgcaaagatg

SEQ ID NO: 64 - RNA, 21 nt, Homo sapiens
  source 1..21: mol_type = other RNA
attaaatgaacacgcaaagat

SEQ ID NO: 65 - RNA, 21 nt, Homo sapiens
  source 1..21: mol_type = other RNA
tattaaatgaacacgcaaaga

SEQ ID NO: 66 - RNA, 21 nt, Homo sapiens
  source 1..21: mol_type = other RNA
ttattaaatgaacacgcaaag

SEQ ID NO: 67 - RNA, 21 nt, Homo sapiens
  source 1..21: mol_type = other RNA
attattaaatgaacacgcaaa

SEQ ID NO: 68 - RNA, 21 nt, Homo sapiens
  source 1..21: mol_type = other RNA
cattattaaatgaacacgcaa

SEQ ID NO: 69 - RNA, 21 nt, Homo sapiens
  source 1..21: mol_type = other RNA
ccattattaaatgaacacgca

SEQ ID NO: 70 - RNA, 45 nt, Homo sapiens
  source 1..45: mol_type = mRNA
ggagaacatttcccttatgaacaagaaatcaagttctttgcaaaa

SEQ ID NO: 71 - RNA, 45 nt, Homo sapiens
  source 1..45: mol_type = mRNA
ggagaacatttcccttatgaacgagaaatcaagttctttgcaaaa

SEQ ID NO: 72 - RNA, 21 nt, Homo sapiens
  source 1..21: mol_type = other RNA
aacgagaaatcaagttctttg

SEQ ID NO: 73 - RNA, 21 nt, Homo sapiens
  source 1..21: mol_type = other RNA
gaacgagaaatcaagttcttt

SEQ ID NO: 74 - RNA, 21 nt, Homo sapiens
  source 1..21: mol_type = other RNA
tgaacgagaaatcaagttctt

SEQ ID NO: 75 - RNA, 21 nt, Homo sapiens
  source 1..21: mol_type = other RNA
atgaacgagaaatcaagttct

SEQ ID NO: 76 - RNA, 21 nt, Homo sapiens
  source 1..21: mol_type = other RNA
tatgaacgagaaatcaagttc

SEQ ID NO: 77 - RNA, 21 nt, Homo sapiens
  source 1..21: mol_type = other RNA
ttatgaacgagaaatcaagtt

SEQ ID NO: 78 - RNA, 21 nt, Homo sapiens
  source 1..21: mol_type = other RNA
cttatgaacgagaaatcaagt

SEQ ID NO: 79 - RNA, 21 nt, Homo sapiens
  source 1..21: mol_type = other RNA
ccttatgaacgagaaatcaag

SEQ ID NO: 80 - RNA, 21 nt, Homo sapiens
  source 1..21: mol_type = other RNA
cccttatgaacgagaaatcaa

SEQ ID NO: 81 - RNA, 21 nt, Homo sapiens
  source 1..21: mol_type = other RNA
tcccttatgaacgagaaatca

SEQ ID NO: 82 - RNA, 21 nt, Homo sapiens
  source 1..21: mol_type = other RNA
ttcccttatgaacgagaaatc

SEQ ID NO: 83 - RNA, 21 nt, Homo sapiens
  source 1..21: mol_type = other RNA
tttcccttatgaacgagaaat

SEQ ID NO: 84 - RNA, 21 nt, Homo sapiens
  source 1..21: mol_type = other RNA
atttcccttatgaacgagaaa

SEQ ID NO: 85 - RNA, 21 nt, Homo sapiens
  source 1..21: mol_type = other RNA
catttcccttatgaacgagaa

SEQ ID NO: 86 - RNA, 21 nt, Homo sapiens
  source 1..21: mol_type = other RNA
acatttcccttatgaacgaga

SEQ ID NO: 87 - RNA, 45 nt, Homo sapiens; identical to SEQ ID NO: 70
  source 1..45: mol_type = mRNA

SEQ ID NO: 88 - RNA, 45 nt, Homo sapiens; identical to SEQ ID NO: 71
  source 1..45: mol_type = mRNA

SEQ ID NO: 89 - RNA, 21 nt, Homo sapiens
  source 1..21: mol_type = other RNA
aacaagaaatcaagttctttg

SEQ ID NO: 90 - RNA, 21 nt, Homo sapiens
  source 1..21: mol_type = other RNA
gaacaagaaatcaagttcttt

SEQ ID NO: 91 - RNA, 21 nt, Homo sapiens
  source 1..21: mol_type = other RNA
tgaacaagaaatcaagttctt

SEQ ID NO: 92 - RNA, 21 nt, Homo sapiens
  source 1..21: mol_type = other RNA
atgaacaagaaatcaagttct

SEQ ID NO: 93 - RNA, 21 nt, Homo sapiens
  source 1..21: mol_type = other RNA
tatgaacaagaaatcaagttc

SEQ ID NO: 94 - RNA, 21 nt, Homo sapiens
  source 1..21: mol_type = other RNA
ttatgaacaagaaatcaagtt

SEQ ID NO: 95 - RNA, 21 nt, Homo sapiens
  source 1..21: mol_type = other RNA
cttatgaacaagaaatcaagt

SEQ ID NO: 96 - RNA, 21 nt, Homo sapiens
  source 1..21: mol_type = other RNA
ccttatgaacaagaaatcaag

SEQ ID NO: 97 - RNA, 21 nt, Homo sapiens
  source 1..21: mol_type = other RNA
cccttatgaacaagaaatcaa

SEQ ID NO: 98 - RNA, 21 nt, Homo sapiens
  source 1..21: mol_type = other RNA
tcccttatgaacaagaaatca

SEQ ID NO: 99 - RNA, 21 nt, Homo sapiens
  source 1..21: mol_type = other RNA
ttcccttatgaacaagaaatc

SEQ ID NO: 100 - RNA, 21 nt, Homo sapiens
  source 1..21: mol_type = other RNA
tttcccttatgaacaagaaat

SEQ ID NO: 101 - RNA, 21 nt, Homo sapiens
  source 1..21: mol_type = other RNA
atttcccttatgaacaagaaa

SEQ ID NO: 102 - RNA, 21 nt, Homo sapiens
  source 1..21: mol_type = other RNA
catttcccttatgaacaagaa

SEQ ID NO: 103 - RNA, 21 nt, Homo sapiens
  source 1..21: mol_type = other RNA
acatttcccttatgaacaaga

SEQ ID NO: 104 - RNA, 21 nt, Homo sapiens
  source 1..21: mol_type = other RNA
aacatttcccttatgaacaag

SEQ ID NO: 105 - DNA, 56 bp, Homo sapiens
  source 1..56: mol_type = genomic DNA
aacagaagctgctgaactattttgctcgcaacttttacaacatgagaatgctggcc

SEQ ID NO: 106 - DNA, 56 bp, Homo sapiens
  source 1..56: mol_type = genomic DNA
aacagaagctgctgaactattttgcttgcaacttttacaacatgagaatgctggcc

SEQ ID NO: 107 - RNA, 21 nt, Homo sapiens
  source 1..21: mol_type = other RNA
tgcttgcaacttttacaacat

SEQ ID NO: 108 - RNA, 21 nt, Homo sapiens
  source 1..21: mol_type = other RNA
ttgcttgcaacttttacaaca

SEQ ID NO: 109 - RNA, 21 nt, Homo sapiens
  source 1..21: mol_type = other RNA
tttgcttgcaacttttacaac

SEQ ID NO: 110 - RNA, 21 nt, Homo sapiens
  source 1..21: mol_type = other RNA
ttttgcttgcaacttttacaa

SEQ ID NO: 111 - RNA, 21 nt, Homo sapiens
  source 1..21: mol_type = other RNA
attttgcttgcaacttttaca

SEQ ID NO: 112 - RNA, 21 nt, Homo sapiens
  source 1..21: mol_type = other RNA
tattttgcttgcaacttttac

SEQ ID NO: 113 - RNA, 21 nt, Homo sapiens
  source 1..21: mol_type = other RNA
ctattttgcttgcaactttta

SEQ ID NO: 114 - RNA, 21 nt, Homo sapiens
  source 1..21: mol_type = other RNA
actattttgcttgcaactttt

SEQ ID NO: 115 - RNA, 21 nt, Homo sapiens
  source 1..21: mol_type = other RNA
aactattttgcttgcaacttt

SEQ ID NO: 116 - RNA, 21 nt, Homo sapiens
  source 1..21: mol_type = other RNA
gaactattttgcttgcaactt

SEQ ID NO: 117 - RNA, 21 nt, Homo sapiens
  source 1..21: mol_type = other RNA
tgaactattttgcttgcaact

SEQ ID NO: 118 - RNA, 21 nt, Homo sapiens
  source 1..21: mol_type = other RNA
ctgaactattttgcttgcaac

SEQ ID NO: 119 - RNA, 21 nt, Homo sapiens
  source 1..21: mol_type = other RNA
gctgaactattttgcttgcaa

SEQ ID NO: 120 - DNA, 22 bp, synthetic construct
  source 1..22: mol_type = other DNA; note = synthetic oligonucleotide
ctatatcatggccgacaagcag

SEQ ID NO: 121 - DNA, 21 bp, synthetic construct
  source 1..21: mol_type = other DNA; note = synthetic oligonucleotide
gcgtgatgaacttcgaggacg

SEQ ID NO: 122 - DNA, 20 bp, synthetic construct
  source 1..20: mol_type = other DNA; note = synthetic oligonucleotide
gctcgtccatgccgagcgtg

SEQ ID NO: 123 - DNA, 21 bp, synthetic construct
  source 1..21: mol_type = other DNA; note = synthetic oligonucleotide
cagcccatggtcttcttctgc

SEQ ID NO: 124 - DNA, 19 bp, synthetic construct
  source 1..19: mol_type = other DNA; note = synthetic oligonucleotide
gaacctccagcgatactgc

SEQ ID NO: 125 - DNA, 33 bp, synthetic construct
  source 1..33: mol_type = other DNA; note = synthetic oligonucleotide
ctggtacccttgtcatcgtcatccttgtaatcg

SEQ ID NO: 126 - DNA, 29 bp, synthetic construct
  source 1..29: mol_type = other DNA; note = synthetic oligonucleotide
ctggtaacctattaagcgtagtcaggtac

SEQ ID NO: 127 - DNA, 20 bp, synthetic construct
  source 1..20: mol_type = other DNA; note = synthetic oligonucleotide
aaatcccatcaccatcttcc

SEQ ID NO: 128 - DNA, 20 bp, synthetic construct
  source 1..20: mol_type = other DNA; note = synthetic oligonucleotide
ggttcacacccatgacgaac

SEQ ID NO: 129 - DNA, 31 bp, synthetic construct
  source 1..31: mol_type = other DNA; note = synthetic oligonucleotide
tgctgtaaaagttgcaagcaaaatagttttg

SEQ ID NO: 130 - DNA, 32 bp, synthetic construct
  source 1..32: mol_type = other DNA; note = synthetic oligonucleotide
gccactgactgactattttgcgcaacttttac

SEQ ID NO: 131 - DNA, 31 bp, synthetic construct
  source 1..31: mol_type = other DNA; note = synthetic oligonucleotide
cctggtaaaagttgcgcaaaatagtcagtca

SEQ ID NO: 132 - DNA, 32 bp, synthetic construct
  source 1..32: mol_type = other DNA; note = synthetic oligonucleotide
gtggccaaaactattttgcttgcaacttttac

SEQ ID NO: 133 - DNA, 25 bp, synthetic construct
  source 1..25: mol_type = other DNA; note = synthetic oligonucleotide
tagctagctgcttcgcgatgtacgg

SEQ ID NO: 134 - DNA, 29 bp, synthetic construct
  source 1..29: mol_type = other DNA; note = synthetic oligonucleotide
gtgaattcgaacaaacgacccaacacccg